SEQUENCE LISTING (29 sequences)

SEQ ID NO: 1 - DNA, 2169 bp, Campylobacter jejuni
  source 1..2169: mol_type = other DNA
atgttgaaaaaagagtatttaaaaaacccttatttagttttgtttgcgatgattatattagcttatgtttttagtgtattttgcaggttttattgggtttggtgggcaagtgagtttaatgagtattttttcaataatcagttaatgatcatttcaaatgatggctatgcttttgctgagggcgcaagagatatgatagcaggttttcatcagcctaatgatttgagttattatggatcttctttatccgcgcttacttattggctttataaaatcacacctttttcttttgaaagtatcattttatatatgagtacttttttatcttctttggtggtgattcctactattttgctagctaacgaatacaaacgtcctttaatgggctttgtagctgctcttttagcaagtatagcaaacagttattataatcgcactatgagtgggtattatgatacggatatgctggtaattgttttgcctatgtttattttattttttatggtaagaatgattttaaaaaaagactttttttcattgattgccttgccgttatttataggaatttatctttggtggtatccttcaagttatactttaaatgtagctttaattggactttttttaatttatacacttatttttcatagaaaagaaaagattttttatatagctgtgattttgtcttctcttactctttcaaatatagcatggttttatcaaagtgccattatagtaatactttttgctttattcgccttagagcaaaaacgcttaaattttatgattataggaattttaggtagtgcaactttgatatttttgattttaagtggtggggttgatcctatactttatcagcttaaattttatatttttagaagtgatgaaagtgcgaatttaacgcagggctttatgtattttaatgtcaatcaaaccatacaagaagttgaaaatgtagatcttagcgaatttatgcgaagaattagtggtagtgaaattgtttttttgttttctttgtttggttttgtatggcttttgagaaaacataaaagtatgattatggctttacctatattggtgcttgggtttttagccttaaaaggggggcttagatttaccatttattctgtacctgtaatggccttaggatttggttttttattgagcgagtttaaggctataatggttaaaaaatatagccaattaacttcaaatgtttgtattgtttttgcaactattttgactttagctccagtatttatccatatttacaactataaagcgccaacagttttttctcaaaatgaagcatcattattaaatcaattaaaaaatatagccaatagagaagattatgtggtaacttgggcggcttatggttatcctgtgcgttattatagcgatgtgaaaactttagtagatggtggaaagcatttaggtaaggataattttttcccttcttttgctttaagcaaagatgaacaagctgcagctaatatggcaagacttagtgtagaatatacagaaaaaagcttttatgctccgcaaaatgatattttaaaaacagacattttgcaagccatgatgaaagattataatcaaagcaatgtggatttgtttctagcttcattatcaaaacctgattttaaaatcgatacgccaaaaactcgtgatatttatctttatatgcccgctagaatgtctttgattttttctacggtggctagtttttcttttattaatttagatacaggagttttggataaaccttttacctttagcacagcttatccacttgatgttaaaaatggagaaatttatcttagcaacggagtggttttaagcgatgattttagaagttttaaaataggtgataatgtggtttctgtaaatagtatcgtagagattaattctattaaacaaggtgaatacaaaatcactccaattgatgataaggctcagttttatattttttatttaaaggatagtgctattccttacgcacaatttattttaatggataaaaccatgtttaatagtgcttatgtgcaaatgttttttttaggaaattatgataagaatttatttgacttggtgattaattctagagatgctaaggtttttaaacttaaaatttacccatacgatgttccagattacgcttaa

SEQ ID NO: 2 - protein, 722 aa, Campylobacter jejuni
MLKKEYLKNPYLVLFAMIILAYVFSVFCRFYWVWWASEFNEYFFNNQLMIISNDGYAFAEGARDMIAGFHQPNDLSYYGSSLSALTYWLYKITPFSFESIILYMSTFLSSLVVIPTILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAIMVKKYSQLTSNVCIVFATILTLAPVFIHIYNYKAPTVFSQNEASLLNQLKNIANREDYVVTWAAYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKIYPYDVPDYA

SEQ ID NO: 3 - DNA, 28 bp, synthetic construct
  misc_feature 1..28: note = Description of Artificial Sequence: Synthetic oligonucleotide
  source 1..28: mol_type = other DNA
aattctgcaggatcctctagaagcttgg

SEQ ID NO: 4 - DNA, 28 bp, synthetic construct
  misc_feature 1..28: note = Description of Artificial Sequence: Synthetic oligonucleotide
  source 1..28: mol_type = other DNA
aattccaagcttctagaggatcctgcag

SEQ ID NO: 5 - DNA, 10174 bp, unidentified
  misc_feature 1..10174: note = source = /note="Description of Unknown: Shigella dysenteriae O1 Antigen in the PGVXN64 plasmid"
  source 1..10174: mol_type = other DNA
atgaaaaaccataaggttagtattatcatcccctgttttaataacggaaaaactatagaaagatgcgttctttccgcattaaaacaaacccatagaaatatagagattatttgtataaatgatggaccgtcagataatagctggaatattttaaagaaattaagctcattgtatggaaatgtttttgcttttaataatgaggacaattcagggccgtcattttccaggattaaaggggtttcactatctacggggcattttttgtcatttttggatgcagatgattactggcatccaaaaaaactagaattacaactatcatttattaatgatgaaaacttggattttttaggttcaacgtgttccattggtgagaaaaataaccaagaaattaaccaaggaattaaaaaagaacatttaaaattaaaaataatttcatttaacatgatgttgtttaagaattatttccagactccagctgtcattatgaaaagagatatttttattccatttaatgagaatcagcgtttttcagaggactacatgtcatggcttgttatcgtttataataaaaaaacaaatgtggatttaatatatggaagggatttggtttttctcgataaatttaactttggagtgtcagggttgagtggtaatttatggttgatggagaagtgggagttaaaaaatatatttaacttcttgttgaaaggtaaaataatggcagtgcctgcgatcttgttttctttgataaaatatgaaagaagatgcgctttaacaaagaaaaataaaggtaagggtaataaataatgaagatctcaataatagggaacacagcaaatgctatgattttgtttagattggatttaataaaaacactaaccaacaaagggatttcagtctatgcttttgctactgactataatgattcatccaaggaaataataaaaaaagcaggcgccattcctgttgattataatttaagtcgcagtggtattaaccttgctggtgatttatggaatacttacttattaagtaaaaaactaaagaagataaaaccagatgctattttatcttttttttcaaagccctctatctttggatcgttggctggtattttttcaggcgttaaaaataataacgctatgcttgaggggttaggttttttatttacagagcagccacatggaactccgttaaaaacaaagttacttaaaaatatccaggttctcctgtataaaataatatttccacatatcaactcattaatactccttaacaaggatgattatcatgatttgatagataaatacaaaataaaattaaaatcttgccatattcttggtggcattggtttagatatgaataattactgtaaaagcacgccaccaacaaatgaaatatcattcatttttatagctcgtttgctagcagaaaaaggagtcaatgagtttgttgctgccgcaaaaaaaataaaaaaaacacatcccaatgttgaatttattatacttggcgctatagataaggaaaaccccggagggttatctgaatctgacgtagatactttaattaaatcaggagttatttcttatcccggatttgtttctaatgtggctgattggattgaaaaatcaagcgtatttgttcttccttcctattatcgagagggagttcctcgtagtacacaagaagcgatggctatggggaggccgattttaactactaatttaccaggctgcaaagaaacaattattgatggtgtgaatggatatgttgtaaaaaaatggtcacatgaagatcttgcagaaaaaatgctgaagttaattaataatcctgaaaaaataatcagtatgggagaagaaagttataagttagcaagagaaagattcgatgcaaatgtaaataatgtaaagttattaaaaatactagggattcctgattaataaacgaaaagcggctctgattcattcggaactaagaacctatctcaataggagctaaattcatgaccttacccagccatatcgacctgcagcctgactgatgccattgccgaactggcgaaaaaacagtccgttgatgccatgctgatgactggagacagctacgactgcggaaaaaaaatgggctatatgcaggcgtttgtgaagtatgggctgcgcaacctcaaagaaggggcgaagttccgtaaagggattgagaagctgttaagcgaataatgaaaatctgaccgaatgtaacggttgataagaaaattataacggcagtgaagattcgtggcgaaagtaatttgttgcgaatattcctgccgttgttttatataaacaatcagaataacaaagagttagcaataggattttcgtcaaagttttccaggattttccttgtttccagagcggattggtaagacaattagtgtttgaatttttcgggtttagcgcgagtgggtaacgctcgtcacatcgtggacatgtatgcagtgctctggtagctgtaaagccaggggcggtagcgtgcattaatacctctattaatcaaactgagagccgcttatttcacagcatgctctgaagtaatatggaataataaagtgaagatacttgttactggtggcgcaggatttattggttctgctgtagttcgtcacattataaataatacgcaggatagtgttgttaatgtcgataaattaacgtacgccggaaacctggagtcacttgctgatgtttctgactctaaacgctatgtttttgaacatgcggatatttgcgatgctgctgcaatggcgcggatttttgctcagcatcagccggatgcagtgatgcacctggctgctgaaagccatgtggatcgttcaattacaggccctgcggcatttattgaaaccaatattgttggtacttatgtccttttggaagcggctcgcaattactggtctgctcttgatggcgacaagaaaaatagcttccgttttcatcatatttctactgacgaagtctatggtgatttgcctcatcctgacgaagtaaataataaagaacaattacccctctttactgagacgacagcttacgcgcctagtagtccttattccgcatcaaaagcatccagcgatcatttagtccgtgcgtggaaacgtacctatggtttaccgaccattgtgactaactgttcgaataactacggtccttatcactttccggaaaaattgattccactagtaattcttaatgctctggaaggtaaggcattacctatttatggcaaaggggatcaaattcgtgactggctgtatgttgaagatcatgcgcgtgcgttatatatcgtcgtaaccgaaggtaaagcgggtgaaacttataacattggtggacacaacgaaaagaaaaacatcgatgtagtgctcactatttgtgatttgttggatgagattgtaccgaaagagaaatcttaccgcgagcaaattacttatgttgccgatcgcccgggacacgatcgccgttatgcgattgatgcagagaagattagccgcgaattgggctggaaaccgcaggaaacgtttgagagcgggattcgtaaaacggtgggatggtacctctccaatacaaaatgggttgataatgtaaaaagtggtgcctatcaatcgtggattgaacagaactatgagggccgccagtaatgaatatcctccttttcggcaaaacagggcaggtaggttgggaactacagcgtgctctggcacctctgggtaatttgattgctcttgatgttcactccactgattactgtggtgattttagtaatcctgaaggtgtagctgaaaccgtaagaagcattcggcctgatattattgtcaacgcagccgctcacaccgcagtagacaaagcagaatcagaaccggagtttgcacaattacttaacgcgacgagtgtcgaagcgatcgcgaaagcagccaatgaagtcggcgcctgggttattcactactctactgactacgtatttccggggaccggtgaaataccatggcaggaggcggatgcaaccgcaccgctaaatgtttacggtgaaaccaagttagctggagaaaaagcattacaagagcattgtgcgaagcacctaattttccgtacaagctgggtctatgcaggtaaaggaaataacttcgccaaaacgatgttgcgtctgggaaaagagcgtgaagaattagccgttattaatgatcagtttggtgcgccaacaggtgctgaactgctggctgattgtacggcacatgcaattcgtgtggcactgaataaaccagaagtcgcaggcttgtaccatctggtagccactggtaccacaacctggcacgattatgctgcgctggtttttgaagaggcacgaaaagcaggtattccccttgcactcaacaagctcaacgcagtaccaacaacagcttatcctacaccagctcgtcgtccacataactctcgccttaatacagaaaaatttcagcaaaattttgcgcttgttttgcctgactggcaggttggcgtgaaacgaatgctcaacgaattatttacgactacagcaatttaatagtttttgcatcttgttcgtgatgatggagcaagatgaattaaaaggaatgatgtaatgaaaacgcgtaaaggtattattttagcgggtggctctggtactcgtctttatcctgtgactatggctgtcagtaaacagctattacctatttatgataagccgatgatctattacccgctctctacactgatgttggcgggtattcgcgatattctgattattagtacgccacaggatactcctcgttttcaacaactcctgggtgatggtagccagtgggggttaaatcttcagtacaaagtgcaaccgagtccagatggtcttgcgcaggcatttatcatcggtgaagagtttatcggtggtgatgattgtgctctggttctcggtgataatatcttctacggtcatgatctgccgaagttaatggatgtcgctgtcaacaaagaaagtggtgcaacggtatttgcctatcacgttaatgatcctgaacgctacggtgttgttgagtttgataaaaacggtacggcaatcagcctggaagaaaaaccgctacaaccaaaaagtaattatgcggtaaccgggctttatttctatgataacgacgttgtcgaaatggcgaaaaaccttaagccttctgcccgtggtgaactggaaattaccgatattaaccgtatttatatggagcaggggcgtttatccgttgccatgatgggacgtggttatgcatggctggacacggggacacatcaaagtcttattgaagcaagcaacttcattgcaacaattgaagagcgccaagggttaaaggtatcttgcctggaagagattgcttatcgtaaaggctttattgacgcagagcaggttaatgtattagccgaaccgctaaagaaaaatgcttatggtcagtatctgttgaaaatgattaaaggttattaaaaatgaatgtaattaaaactgaaattccagatgtattaattttcgagccgaaagtttttggtgatgaacgtggtttttttatggaaagctttaaccagaaagttttcgaagaggctgtagggcggaaggttgaatttgttcaggataaccattctaaatcaactaagggtgtgttacgcggactgcactatcagttggaaccttatgctcaaggtaaattagttcgttgtgttgtcggtgaagtttttgatgtagcagttgatattcgtaaatcgtcacctacatttgggaaatggattggggtgaatttgtctgctgagaataagcgtcagttgtggatacctgaaggatttgcgcatggatttttggtgctgagtgaaacggctgagtttgtttataaaacaacaaactattacaatccaagttttgaaaaaagtatttcatactcagatcctaccattaaaattcagtggcccaatttacaggatatgcattttaaattatcaaataaggatttgaatgctaagaacttttttaataacaatagtttaatgcaatgaagaaaaatatattgctcttgttcttagtacatggggcaaattatttgttcccgtttatagttcttccatatcaaactcgaatattaagcatcgagacattcgcagatgtagcaaaaattcaagccgctgtgatgcttttatctttaatcgtaaattatggatataacttatcaagtacaagagctatagctagggccgtatctcaagcagaaataaataagatctatagtgagactcttattgtaaaattattattggcaaccatttgtcttgcacttggttgcgtacatttgatgtatgtcaaagagtactcattgatatatccttttataatcagttcgatatatctttatggtagtgcattatttgctacttggttattccaaggacttgagaaaatgaaagcggtcgttatagcaacaacaatcgctaaactgactggtgtgatacttacttttattttagttaagtctccaaatgatatagttgcagctctttttacacaaaacattgggatgtttataagtggtataatatctatttatttggtaaggaaaaacaaatatgcaaccgtaatatgttttcgacttaaaaatattattgtaagcttaaaagaagcgtggccgttttttttatcattagctgcaacaagtgtatatacatattttaatgtgattttattatctttttatgctggcgactatgttgtggcaaattttaatgctgctgataaattaagaatggctgctcaagggttacttattccaataggacaggctgttttcccacgattatctaaactagagggctatgaatatagttctaaacttaaaatttatgcaataaggtatgctatttttggtgtttgcattagtgcgggacttgtatttttaggtcccatgttaactactatttatttaggcaaagaatattcgttgtcaggagaatatcttcaaagtatgtttttactacctgccactatttcaatatcgactatactgagtcaatggatgttgatacctcaaggcaaagaaaaaatattaagcagaatctatattctaggcgccattgtccatttattatatgcatttcctttagtttactattatggggcttggggcatggtaatatcaattttatttactgaagtcttaattgtattatttatgcttaaggctgtgaaatgacttactttactggttttattttaatattgtttgctattataattaaaagattaactccaagtcaaagcaagaaaaatattgtcttaatagctaatgcgttttggggaatattgttggtaggttatgctttcaatgaacaatatttcgtaccattaagtgcaacaaccttgttttttatacttgcattcttatttttctttagtatgacttatattttaattgctaggagtggaagggttgttttttctttcggtactggttttatagaaagcaaatatatttactggtttgctgggatgattaatattattagtatctgctttggcattatccttttatataataatcatttttctttaaaagtaatgagagaaggaattttagatggttctattagtgggtttggattggggataagtttgccactttccttctgctgtatgtatttagcaagacatgagaataaaaaaaattatttctattgttttacactactttcattcttgcttgcggtgttatcaacttcaaagatcttcttaatattattccttgtatatattgttggaataaatagttatgtaagcaaaaagaaattgcttatttatggagtgtttgtatttggactgttcgctttatcaagtattatcttgggtaagttctcttcagaccctgaaggcaagattatttcagcaatatttgatacgttaagggtttatcttttctcgggattggcagcctttaatctttatgttgaaaagaatgccacgctccccgaaaatttacttttgtatccatttaaggaggtttgggggacgacaaaagatattcccaaaactgatattttgccttggatcaacattggtgtatgggacacgaatgtatatacagcttttgcaccatggtatcagtcattgggattatatgcagctataattattggtattctcttagggttttattacgggatatggtttagctttcgtcaaaatttagctgtgggtttttatcaaacatttttgtgttttcctcttttaatgttgtttttccaggagcattatttgttgtcatggaaaatgcattttatttattttttatgtgcaattttattagcgatgagaaaagcattagagtatgaataaatattgtatcttagtactatttaatccagatataagtgtttttattgataatgtcaaaaagattttatctttggatgtaagtttatttgtatatgacaattcagcaaataaacatgcattccttgctctatcctcacaagagcaaacaaagataaattacttttcgatatgtgaaaatatcggattgtcgaaagcttataatgagacactaaggcatattcttgaatttaataagaatgtgaaaaataaaagcattaatgatagtgtgctttttctcgaccaagactctgaagttgatttaaattccatcaatattttgtttgaaactatatcagcagcagagtctaatgtgatgatagtcgcggggaatcccataaggagagatggactaccgtatatagattacccccacactgtaaacaatgtaaaatttgtaattagtagttatgctgtgtatcgcttagacgcatttagaaacatcggcttgtttcaagaagatttttttatagatcatatcgatagtgatttttgttcaaggctgataaaaagcaattaccaaattctccttagaaaagatgcctttttttatcaaccaataggaataaaaccattcaatctctgtggtagatatttattccctatcccatcacaacaccgaacatattttcaaattagaaatgcttttttaagttacaggcgcaatggtgttacatttaattttttatttagggaaattgtaaatagattgattatgagtatattctcaggccttaacgagaaagacttattgaaacgattgcatttatatttaaaaggaataaaagatggtcttaaaatgtaattcttggctagaagtgggggcgttgtgattaaaaaaaaagtggcggcgataattataacatataatccagatctaacaattctgcgagaaagttatacgagtctatataagcaagtcgataaaataattcttattgataacaactctacaaactatcaagaacttaagaagttattcgaaaaaaaagaaaaaataaaaatagtgcccttgagtgataatataggactagcagcagctcaaaatttaggtttgaacttagctattaaaaataactatacttatgctattttattcgatcaggatagcgtcttacaagacaatggaattaacagtttcttttttgaatttgagaaattagttagtgaagaaaaattaaatatagttgccattgggccaagtttttttgacgaaaagacaggaagacgctttcggcctacaaaatttatcggtccctttttatatccctttcgtaaaataaccacaaaaaatcctctaacagaagttgacttcttgattgcttctggttgtttcataaaattggagtgtattaaatcagccggaatgatgactgaatcgttattcatcgattatattgatgttgaatggtcatatcgtatgcgttcgtatggctataagctatatattcataatgatattcacatgagtcatttagtgggagaatctcgagttaatttaggattgaaaactatttctttacatgggccgctaagacgatattacttatttaggaattatatttcaattttaaaagtgagatatataccgttaggatataaaatacgtgagggtttttttaatatcggaagatttttggtaagtatgattataactaaaaatagaaaaactttaattttatacactataaaagcaattaaggacggaataaataatgaaatggggaaatataaaggctaa

SEQ ID NO: 6 - protein, 643 aa, synthetic construct
  REGION 1..643: note = Description of Artificial Sequence: Synthetic polypeptide
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 7 - protein, 624 aa, synthetic construct
  REGION 1..624: note = Description of Artificial Sequence: Synthetic polypeptide
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 8 - protein, 123 aa, synthetic construct
  REGION 1..123: note = Description of Artificial Sequence: Synthetic polypeptide
MKKTAIAIAVALAGFATVAQADVIIKPQVSGVIVNKLFKAGDKVKKGQTLFIIEQDQASKDFNRSKALFSQLDHTEIKAPFDGTIGDALVNIGDYVSASTTELVRVTNLNPIYADGSHHHHHH

SEQ ID NO: 9 - protein, 126 aa, synthetic construct
  REGION 1..126: note = Description of Artificial Sequence: Synthetic polypeptide
MKKTAIAIAVALAGFATVAQADVIIKPQVSGVIVNKLFKAGDKVKKGQTLFIIEQDQFNRSKALFSQSAISQKELDHTEIKAPFDGTIGDALVNIGDYVSASTTELVRVTNLNPIYADGSHHHHHH

SEQ ID NO: 10 - protein, 116 aa, synthetic construct
  REGION 1..116: note = Description of Artificial Sequence: Synthetic polypeptide
MKKTAIAIAVALAGFATVAQADVIIKPQVSGVIVNKLFKAGDKVKKGQTLFIIEQDQDFNRSKALDHTEIKAPFDGTIGDALVNIGDYVSASTTELVRVTNLNPIYADGSHHHHHH

SEQ ID NO: 11 - protein, 379 aa, synthetic construct
  REGION 1..379: note = Description of Artificial Sequence: Synthetic polypeptide
MKYLLPTAAAGLLLLAAQPAMAMHMSKEEAPKIQMPPQPVTTMSAKSEDLPLSFTYPAKLVSDYDVIIKPQVSGVIVNKLFKAGDKVKKGQTLFIIEQDKFKASVDSAYGQALMAKATFENASKDFNRSKALFSKSAISQKEYDSSLATFNNSKASLASARAQLANARIDLDHTEIKAPFDGTIGDALVNIGDYVSASTTELVRVTNLNPIYADFFISDTDKLNLVRNTQSGKWDLDSIHANLNLNGETVQGKLYFIDSVIDANSGTVKAKAVFDNNNSTLLPGAFATITSEGFIQKNGFKVPQIGVKQDQNDVYVLLVKNGKVEKSSVHISYQNNEYAIIDKGLQNGDKIILDNFKKIQVGSEVKEIGAQLEHHHHHH

SEQ ID NO: 12 - protein, 132 aa, synthetic construct
  REGION 1..132: note = Description of Artificial Sequence: Synthetic polypeptide
MKKTAIAIAVALAGFATVAQATPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGSHHHHHH

SEQ ID NO: 13 - protein, 288 aa, Campylobacter jejuni
MKKILLSVLTTFVAVVLAACGGNSDSKTLNSLDKIKQNGWRIGVFGDKPPFGYVDEKGNNQGYDIALAKRIAKELFGDENKVQFVLVEAANRVEFLKSNKVDIILANFTQTPERAEQVDFCLPYMKVALGVAVPKDSNITSVEDLKDKTLLLNKGTTADAYFTQDYPNIKTLKYDQNTETFAALMDKRGDALSHDNTLLFAWVKDHPDFKMGIKELGNKDVIAPAVKKGDKELKEFIDNLIIKLGQEQFFHKAYDETLKAHFGDDVKADDWIEGGKILEQKLISEEDL

SEQ ID NO: 14 - DNA, 30 bp, synthetic construct
  misc_feature 1..30: note = Description of Artificial Sequence: Synthetic oligonucleotide
  source 1..30: mol_type = other DNA
aagctagcgccgccgaggaagccttcgacc

SEQ ID NO: 15 - DNA, 50 bp, synthetic construct
  misc_feature 1..50: note = Description of Artificial Sequence: Synthetic oligonucleotide
  source 1..50: mol_type = other DNA
aagaattctcagtggtggtggtggtggtgcttcaggtcctcgcgcggcgg

SEQ ID NO: 16 - DNA, 27 bp, synthetic construct
  misc_feature 1..27: note = Description of Artificial Sequence: Synthetic oligonucleotide
  source 1..27: mol_type = other DNA
gaaggcgggcgcgtgaccattctcggc

SEQ ID NO: 17 - DNA, 27 bp, synthetic construct
  misc_feature 1..27: note = Description of Artificial Sequence: Synthetic oligonucleotide
  source 1..27: mol_type = other DNA
gccgagaatggtcacgcgcccgccttc

SEQ ID NO: 18 - DNA, 26 bp, synthetic construct
  misc_feature 1..26: note = Description of Artificial Sequence: Synthetic oligonucleotide
  source 1..26: mol_type = other DNA
cctgacctgccccggggaatgcgcgg

SEQ ID NO: 19 - DNA, 26 bp, synthetic construct
  misc_feature 1..26: note = Description of Artificial Sequence: Synthetic oligonucleotide
  source 1..26: mol_type = other DNA
ccgcgcattccccggggcaggtcagg

SEQ ID NO: 20 - DNA, 27 bp, synthetic construct
  misc_feature 1..27: note = Description of Artificial Sequence: Synthetic oligonucleotide
  source 1..27: mol_type = other DNA
gtcgccaaagatcaaaatagaactaaa

SEQ ID NO: 21 - DNA, 27 bp, synthetic construct
  misc_feature 1..27: note = Description of Artificial Sequence: Synthetic oligonucleotide
  source 1..27: mol_type = other DNA
tttagttctattttgatctttggcgac

SEQ ID NO: 22 - DNA, 55 bp, synthetic construct
  misc_feature 1..55: note = Description of Artificial Sequence: Synthetic oligonucleotide
  source 1..55: mol_type = other DNA
catgacctggacatcaaggataataataattctactcccacggtcatcagtcatc

SEQ ID NO: 23 - DNA, 55 bp, synthetic construct
  misc_feature 1..55: note = Description of Artificial Sequence: Synthetic oligonucleotide
  source 1..55: mol_type = other DNA
gatgactgatgaccgtgggagtagaattattattatccttgatgtccaggtcatg

SEQ ID NO: 24 - protein, 6 aa, synthetic construct
  REGION 1..6: note = Description of Artificial Sequence: Synthetic 6xHis tag
HHHHHH

SEQ ID NO: 25 - protein, 5 aa, synthetic construct
  REGION 1..5: note = Description of Artificial Sequence: Synthetic peptide
DSNIT

SEQ ID NO: 26 - protein, 5 aa, synthetic construct
  REGION 1..5: note = Description of Artificial Sequence: Synthetic peptide
HFGDD

SEQ ID NO: 27 - protein, 5 aa, synthetic construct
  REGION 1..5: note = Description of Artificial Sequence: Synthetic peptide
DFNNS

SEQ ID NO: 28 - protein, 5 aa, synthetic construct
  REGION 1..5: note = Description of Artificial Sequence: Synthetic peptide
DNNST

SEQ ID NO: 29 - protein, 5 aa, synthetic construct
  REGION 1..5: note = Description of Artificial Sequence: Synthetic peptide
DNNKT

(19) United States
(12) Patent Application Publication
(10) Pub. No.: US 2024/0277824 A1
(43) Pub. Date: Aug. 22, 2024

(54) BIOCONJUGATES MADE FROM RECOMBINANT N-GLYCOSYLATED PROTEINS FROM PROCARYOTIC CELLS

(71) Applicant: GLAXOSMITHKLINE BIOLOGICALS SA, RIXENSART (BE)

(72) Inventors: Fabiana FERNANDEZ, Unterengstringen (CH); Michael KOWARIK, Zurich (CH); Michael WACKER, Unterengstringen (CH); Michael WETTER, Zurich (CH)

(73) Assignee: GLAXOSMITHKLINE BIOLOGICALS SA, RIXENSART (BE)

(21) Appl. No.: 18/402,174

(22) Filed: Jan. 2, 2024

Related U.S. Application Data
(62) Division of application No. 17/061,687, filed on Oct. 2, 2020, now Pat. No. 11,944,675.
Application No. 17/061,687 is a continuation of application No. 14/522,254, filed on Oct. 23, 2014, now Pat. No. 10,835,592.
Application No. 14/522,254 is a continuation of application No. 12/735,773, filed on Dec. 14, 2010, now Pat. No. 8,895,014.
(60) Provisional application No. 61/064,163, filed on Feb. 20, 2008.
(60) Provisional application No. 61/071,545, filed on May 5, 2008.
(60) Provisional application No. 61/129,480, filed on Jun. 30, 2008.
(60) Provisional application No. 61/129,852, filed on Jul. 24, 2008.
(60) Provisional application No. 61/136,687, filed on Sep. 25, 2008.

(51) Int. Cl. A61K 39/112; A61K 39/00; A61K 39/02; A61K 39/104; A61K 39/108; A61K 39/385; C07K 14/195

(52) U.S. Cl. CPC A61K 39/0283; A61K 39/0258; A61K 39/104; A61K 39/105; A61K 39/385; C07K 14/195; A61K 2039/6037; A61K 2039/6068; A61K 2039/6087; C07K 2319/034; Y02A 50/30

(57) ABSTRACT

The present invention is directed to a bioconjugate vaccine, such as an O 1-bioconjugate vaccine, comprising: a protein carrier comprising a protein carrier containing at least one consensus sequence, DIE-X-N-Z-S/T, wherein X and Z may be any natural amino acid except proline; at least one antigenic polysaccharide from at least one pathogenic bacterium, linked to the protein carrier; and, optionally, an adjuvant. In another aspect, the present invention is directed to a method of producing an O 1-bioconjugate in a bioreactor comprising a number steps.

### CROSS-REFERENCE TO RELATED APPLICATIONS

This application is a Divisional of copending application Ser. No. 17/061,687, filed on Oct. 2, 2020, which is a continuation of U.S. patent application Ser. No. 14/522,254, filed Oct. 23, 2014 (now U.S. Pat. No. 10,835,592, issued Nov. 17, 2020), which is a continuation of U.S. patent application Ser. No. 12/735,773, filed Dec. 14, 2010 (now U.S. Pat. No. 8,895,014, issued Nov. 25, 2014), which is a U.S. national stage entry of International Patent Application No. PCT/IB2009/000287, filed Feb. 19, 2009, which claims priority under 35 U.S.C. § 119(e) to U.S. Provisional Application No. 61/064,163, filed on Feb. 20, 2008, U.S. Provisional Application No. 61/071,545, filed on May 5, 2008, U.S. Provisional Application No. 61/129,480, filed on Jun. 30, 2008, U.S. Provisional Application No. 61/129,852, filed on Jul. 24, 2008, U.S. Provisional Application No. 61/136,687, filed on Sep. 25, 2008, all of which are hereby expressly incorporated by reference into the present application.

### REFERENCE TO SEQUENCE LISTING

The instant application contains a Sequence Listing which has been submitted electronically in XML file format and is hereby incorporated by reference in its entirety. Said XML copy, created on Apr. 9, 2024, is named LB65502C2D1_US_SEQLST.xml and is 53,248 bytes in size.

### FIELD OF THE INVENTION

The present invention relates to bioconjugates, specifically bioconjugate vaccines, made from recombinant glycoproteins, namely N-glycosylated proteins. The invention comprises one or more introduced N-glycosylated proteins with optimized amino acid consensus sequence(s), nucleic acids encoding these proteins as well as corresponding vectors and host cells. In addition, the present invention is directed to the use of said proteins, nucleic acids, vectors and host cells for preparing bioconjugate vaccines. Furthermore, the present invention provides methods for producing bioconjugate vaccines.

### BACKGROUND OF THE INVENTION

Glycoproteins are proteins that have one or more covalently attached sugar polymers. N-linked protein glycosylation is an essential and conserved process occurring in the endoplasmic reticulum of eukarotic organisms. It is important for protein folding, oligomerization, stability, quality control, sorting and transport of secretory and membrane proteins (Helenius, A., and Aebi, M. (2004). Roles of N-linked glycans in the endoplasmic reticulum. Annu. Rev. Biochem. 73, 1019-1049).

Protein glycosylation has a profound influence on the antigenicity, the stability and the half-life of a protein. In addition, glycosylation can assist the purification of proteins by chromatography, e.g. affinity chromatography with lectin ligands bound to a solid phase interacting with glycosylated moieties of the protein. It is therefore established practice to produce many glycosylated proteins recombinantly in eukaryotic cells to provide biologically and pharmaceutically useful glycosylation patterns.

It has been demonstrated that a bacterium, the food-borne pathogen Campylobacter jejuni, can also N-glycosylate its proteins (Szymanski, et al. (1999). Evidence for a system of general protein glycosylation in Campylobacter jejuni. Mol. Microbiol. 32, 1022-1030). The machinery required for glycosylation is encoded by 12 genes that are clustered in the so-called pgl locus.

Disruption of N-gylcosylation affects invasion and pathogenesis of C. jejuni but is not lethal as in most eukaryotic organisms (Burda P. and M. Aebi, (1999). The dolichol pathway of N-linked glycosylation. Biochim Biophys Acta 1426(2):239-57). It is possible to reconstitute the N-glycosylation of C. jejuni proteins by recombinantly expressing the pgl locus and acceptor glycoprotein in E. coli at the same time (Wacker et al. (2002). N-linked glycosylation in Campylobacter jejuni and its functional transfer into E. coli. Science 298, 1790-1793).

Diarrheal illness is a major health problem associated with international travel in terms of frequency and economic impact. Traveller's diarrhea refers to an enteric illness acquired when a person travels from a developed to a developing country. Today, over 50 million people travel each year from developed countries to developing countries and up to 50% of these travelers report having diarrhea during the first 2 weeks of their week of their stay. There has been no significant decline in the incidence of traveller's diarrhea since the 1970s, despite efforts made by the tourism industry to improve local infrastructure.

Traveller's diarrhea is acquired through the ingestion of faecally contaminated food and less commonly water. Bacteria are the main cause of traveller diarrhea's, being responsible for up to 80% of the infections. Enterotoxigenic E. coli (ETEC) is the most frequently isolated bacterium in all parts of the world associated with traveler's diarrhea, followed by Shigella spp and C. jejuni.

Shigellosis remains a serious and common disease. In addition to causing watery diarrhea, Shigellae are a major cause of dysentery (fever, cramps, and blood and/or mucus in the stool). Man is the only natural host for this bacterium. The estimated number of Shigella infections is over 200 million annually. About 5 million of these cases need hospitalization and a million people die. Three serogroups are mostly responsible for the disease described as bacillary dysentery: S. dysenteriae, S. flexneri and S. sonnei.

S. dysenteriae and S. flexneri are responsible for most infections in the tropics, with case fatalities up to 20%. Shigellosis occurs both endemically and as epidemics. In many tropical countries, endemic infection is largely due to S. flexneri whereas major epidemics of S. dysenteriae have occurred in Central America, Central Africa and Southeast Asia. These epidemics are major public-health risks. Infections, primarily due to S. sonnei and less frequently S. flexneri continue to occur in industrialized countries.

Conjugate vaccines have shown promising results against Shigella infections. O-specific polysaccharides of S. dysenteriae type 1 have been used to synthesize a conjugate vaccine that has elicited an immune response in mice. Such vaccines have been synthesized chemically and conjugated to human serum albumin or has been developed where the 0-polysaccharide has been purified from Shigella. The O-specific polysaccharides of S. sonnei and S. βexneri also have been conjugated chemically to P. aeruginosa exotoxin and have elicited a significant immune response in mice. Additionally, they have been shown to be immunogenic and safe in humans. However, chemical conjugation is an expensive and time-consuming process that does not always yield reliable and reproducible vaccines. This leads to good manufacturing practices (GMP) problems when seeking to develop such bioconjugate vaccines on a commercial scale.

### SUMMARY OF THE INVENTION

In one aspect, the present invention is directed to a bioconjugate vaccine comprising: a protein carrier comprising an inserted consensus sequence, D/E-X-N-Z-S/T, wherein X and Z may be any natural amino acid except proline; at least one antigenic polysaccharide from at least one bacterium, linked to the protein carrier, wherein the at least one antigenic polysaccharide is at least one bacterial O-antigen from one or more strains of Shigella, E. coli or Pseudomonas aeruginosa; and, optionally, an adjuvant.

In another aspect, the present invention is directed to a Shigella bioconjugate vaccine comprising: a protein carrier comprising Exotoxin of Pseudomonas aeruginosa (EPA) that has been modified to contain at least one consensus sequence D/E-X-N-Z-S/T, wherein X and Z may be any natural amino acid except proline; at least one polysaccharide chain linked to the protein carrier and having the following structure:

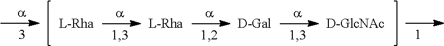

and, optionally, an adjuvant.

In yet another aspect, the present invention is directed to a Shigella dysenteriae OI bioconjugate vaccine comprising: a protein carrier having the sequence provided in SEQ. ID NO.: 7; at least one polysaccharide chain linked to the protein carrier and having the following structure:

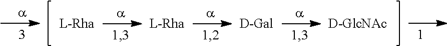

and an adjuvant.

In yet additional aspects, the present invention is directed to: a plasmid comprising SEQ. ID NO. 5; a genetic sequence comprising SEQ. ID NO. 5; an amino acid sequence comprising SEQ. ID NO. 6; an amino acid sequence comprising SEQ. ID NO. 7; or vector pGVXN64.

In another aspect, the present invention is directed to an expression system for producing a bioconjugate vaccine against at least one bacterium comprising: a nucleotide sequence encoding an oligosaccharyl transferase (OST/OTase); a nucleotide sequence encoding a protein carrier; and at least one antigenic polysaccharide synthesis gene cluster from the at least one bacterium, wherein the antigenic polysaccharide is a bacterial O-antigen.

In still another aspect, the present invention is directed to an expression system for producing a bioconjugate vaccine against Shigella dysenteriae OI comprising: a nucleotide sequence encoding PglB having SEQ. ID NO. 2; a nucleotide sequence encoding a modified EPA having SEQ. ID NO. 6; and a polysaccharide synthesis gene cluster comprising SEQ. ID In yet another aspect, the present invention contemplates a method of producing an 01-bioconjugate in a bioreactor comprising the steps: expressing in bacteria: modified EPA containing at least one consensus sequence, D/E-X-N-Z-S/T, wherein X and Z may be any natural amino acid except proline, or AcrA; PglB; and one or more OI-polysaccharides; growing the bacteria for a period of time to produce an amount of the OI-bioconjugate comprising the AcrA or the modified EPA linked to the one more OI-polysaccharides; extracting periplasmic proteins; and separating the 01-bioconjugate from the extracted periplasmic proteins.

In an additional aspect, the present invention contemplates a method of producing an S. dysenteriae bioconjugate vaccine, said method comprising: assembling a polysaccharide of S. dysenteriae in a recombinant organism through the use of glycosyltransferases; linking said polysaccharide to an asparagine residue of one or more target proteins in said recombinant organism, wherein said one or more target proteins contain one or more T-cell epitopes.

In a further aspect, the present invention contemplates a method of producing an S. dysenteriae bioconjugate vaccine, said method comprising: introducing genetic information encoding for a metabolic apparatus that carries out N-glycosylation of a target protein into a prokaryotic organism to produce a modified prokaryotic organism, wherein the genetic information required for the expression of one or more recombinant target proteins is introduced into said prokaryotic organism, and wherein the metabolic apparatus comprises specific glycosyltransferases for the assembly of a polysaccharide of S. dysenteriae on a lipid carrier and an oligosaccharyltransferase, the oligosaccharyltransferase covalently linking the polysaccharide to an asparagine residue of the target protein, and the target protein containing at least one T-cell epitope; producing a culture of the modified prokaryotic organism; and obtaining glycosylated proteins from the culture medium.

### DESCRIPTION OF THE FIGURES

FIG. 1 illustrates the N-glycosylation of Lip proteins derived from constructs A to C (see example 1). E. coli Top 10 cells carrying a functional pgl operon from C. jejuni (Wacker et al., 2002, supra) and a plasmid coding for constructs A (lane 2), B (lane 1), and C (lane 3) or a mutant of construct C with the mutation D121A (lane 4). Proteins were expressed and purified from periplasmic extracts. Shown is the SDS-PAGE and Coomassie brilliant blue staining of the purified protein fractions.

FIG. 2 shows the N-glycosylation analysis of the different proteins that were analyzed for the sequence specific N-glycosylation by the C. jejuni pgl operon (Wacker et al., 2002, supra) in CLM24 cells (Feldman et al., (2005). Engineering N-linked protein glycosylation with diverse O antigen lipopolysaccharide structures in Escherichia coli. Proc. Natl. Acad. Sci. USA 102, 3016-3021) or Top 10 cells (panel E lanes 1-6) or SCM7 cells (Alaimo, C, Catrein, L, Morf, L., Marolda, C. L., Callewaert, N., Valvano, M. A., Feldman, M. F., Aebi, M. (2006). Two distinct but interchangeable mechanisms for flipping of lipid-linked oligosaccharides. EMBO Journal 25, 967-976) (panel E, lanes 7, 8) expressing said proteins from a plasmid. Shown are SDS-PAGE separated periplasmic extracts that were transferred to a nitrocellulose membrane and visualized with specific antisera. In panels A-D, the top panels show immunoblots probed with anti AcrA antiserum (Wacker et al. 2002, supra; Nita-Lazar, M., Wacker, M., Schegg, B., Amber, S., and Aebi, M. (2005). The N-X-S/T consensus sequence is required but not sufficient for bacterial N-linked protein glycosylation. Glycobiology 15, 361-367), whereas the bottom panels show immunoblots probed with R12 antiserum (Wacker et al., 2002, supra). + and − indicate the presence of the functional or mutant pgl operon in the cells. Panel A contains samples of the soluble wildtype AcrA with the pelB signal sequence and the hexa histag (lanes 1, 2), AcrA-N273Q (lane 3, 4), and AcrA-D121A (lane 5). Panel B: AcrA (lanes 1, 2), AcrA-T145D (lane 3), AcrA-N123Q-N273Q-T145D (lanes 4, 5). Panel C: AcrA-FI 15D-T145D (lanes 1, 2), AcrA-N123Q-N273Q-N272D (lanes 3, 4). Panel D: AcrA-N273Q (lanes 1, 2), AcrA-N273Q-F122P (lanes 3, 4). Panel E: CtxB (lanes 1, 2), CtxB-W88D (lanes 3, 4), CtxB-Q56/DSNIT (lanes 5, 6), and CtxB-W88D-Q56/DSNIT.

FIG. 3 shows the engineering of multiple glycosylation sites in OmpHI. The ΔwaaL strain SCM6 was co-transformed with plasmid pACYCpgl (encoding entire pgl locus) and plasmids expressing wild type OmpHI (lane 1), OmpHI^{N139S}-myc (lane 2), OmpHI^{KGN→NIT-HFGDD→DSNIT}_myc (lane 3) QmpH1^{RGD→NIT, HFGD→DSNIT}_myc (lane 4) QmpH1^{KGN→NIT, RGD→NIT}-myc (lane 5)^{KGN-NIT, RGD→N,T HFGDD→DSNIT}-myc (lane 6) or OmpHI^{RGD→NIT-V83T}-myc (lane 7). The cells were grown aerobically, induced with 0.5% arabinose for 3 hours prior to analysis. Whole cell lysates were TCA precipitated after equalizing the optical density of the cultures as described in the materials and methods section. The proteins were separated by 15% SDS-PAGE and transferred onto a PVDF membrane. First panel, immunoblot of whole cell lysates probed with anti-myc tag antibodies. Bottom panel, immunoblot of whole cell lysates probed with glycan-specific antiserum. The positions of unglycosylated- and glycosylated OmpHI are indicated on the right.

FIG. 4. shows fluorescence microscopy of cells expressing various OmpHI variants. Fluorescence microscopy was performed by the using an Axioplan2 microscope (Carl Zeiss). Images were combined by using Adobe Photoshop, version CS2. SCM6 cells expressing OmpHI (panel A), OmpHI^{N139S} (panel B), OmpHI^{C20S} (panel C), OmpHI^{KGN→NΓΓ, HFGDD→DSNIT} (panel D), ompHI^{RGD→NIT_HFGDD→DSNIT} (panel E), OmpHI^{TMN→NIT RGD→NIT} _{(pand Fχ} OmpHI^{V83T KGN→NIT} (panel G), and OmpHI^{KGN→NIT′RGD→NIT′HFGDD→DSNIT} (panel H). The first column is a merge of the pictures in columns 2, 3, and 4 represented in greytones on black background. Column 2: blue fluorescence in greytones from DAPI stain, column 3: green fluorescence from glycan specific fluorescence, column 4: red fluorescence from anti-myc staining.

FIG. 5A shows a schematic of the capsular polysaccharides and lipopolysacharides in Gram-positive and Gram-negative bacteria.

FIG. 5B shows genomic DNA, with integrated PglB and EPA, and plasmid DNA, which is interchangeable (i.e., exchangeable), encoding a polysaccharide synthesis gene cluster.

FIG. 6A shows the production process of conjugate vaccines using technology of the invention.

FIG. 6B shows the construction of the Shigella dysenteriae OI antigen expression plasmid pGVXN64.

FIGS. 7A and 7B show schematics of the protein glycosylation pathway utilized in the present invention.

FIGS. 8A and 8B are schematics depicting expression platforms for bioconjugate production of the present invention.

FIG. 9 shows production of Shigella bioconjugates.

FIG. 10A shows polysaccharide biosynthesis of the O-antigen of S. dysenteriae Serotype OI on undecaprenolpyrophosphate (UPP).

FIG. 10B shows a schematic of a carrier protein, such as EPA, onto which N-glycosylation sites can be designed.

FIG. 11 shows bicoonjugates that elicit an immune response against Shigella OI polysaccharide in mice.

FIG. 12 shows results from the production of a Shigella OI-EPA bioconjugate (e.g., 01-EPA) in a bioreactor.

FIG. 13 shows purification of OI-EPA.

FIG. 14A shows a Western blot analysis of a series of specimens of Shigella OI-AcrA bioconjugates produced in an LB shake flask taken under various conditions.

FIG. 14B provides different serotypes of Shigella and the polysaccharide structure that defines their antigenicity (i.e., Shigella O-antigens).

FIG. 15 shows the expansion of the anomeric region of IH NMR spectrum of an example of a S. dysenteriae Serotype OI bioconjugate of the invention.

FIG. 16A shows protein samples of a Shigella OI Bioconjugate (e.g., EPA-OI) normalized to biomass concentration (0.1 OD₆₀ onm of cells/lane).

FIG. 16B shows the periplasmic extract from a Shigella OI Bioconjugate production process which was loaded on a 7.5% SDS-PAGE, and stained with Coomasie to identify EPA and EPA-OI.

FIG. 17A shows protein fractions from 1. Source Q analyzed by SDS-PAGE and stained by Coomassie to identify the Shigella OI bioconjugate.

FIG. 17B shows protein fractions from 2. Source Q column analyzed on SDS-PAGE and stained by Coomassie to identify the Shigella OI bioconjugate.

FIG. 18A shows protein fractions from Superdex 200 column analyzed by SDS-PAGE and stained by Coomassie stained to identify the Shigella OI bioconjugate.

FIG. 18B shows Shigella bioconjugates from different purification steps analyzed by SDS-PAGE and stained by Coomassie.

### DETAILED DESCRIPTION OF THE INVENTION

### Introduction to Invention

The present invention provides a versatile in vivo glycosylation platform.

European Patent Application No. 03 702 276.1 (European Patent 1 481 057) teaches a procaryotic organism into which is introduced a nucleic acid encoding for (i) specific glycosyltransferases for the assembly of an oligosaccharide on a lipid carrier, (ii) a recombinant target protein comprising a consensus sequence “N-X-S/T”, wherein X can be any amino acid except proline, and (iii) an oligosaccharyl transferase of C. jejuni (OTase) that covalently links said oligosaccharide to the consensus sequence of the target protein. Said procaryotic organism produces N-glycans with a specific structure which is defined by the type of the specific glycosyl transferases.

The known N-glycosylation consensus sequence in a protein allows for the N-glycosylation of recombinant target proteins in procaryotic organisms comprising the oligosaccharyl transferase (OTase) of C. jejuni.

The object of the present invention is to provide proteins as well as means and methods for producing such proteins having an optimized efficiency for N-glycosylation that can be produced in procaryotic organisms in vivo. Another object of the present invention aims at the more efficient introduction of N-glycans into recombinant proteins for modifying antigenicity, stability, biological, prophylactic and/or therapeutic activity of said proteins. A further object is the provision of a host cell that efficiently displays recombinant N-glycosylated proteins of the present invention on its surface.

In a first aspect, the present invention provides a recombinant N-glycosylated protein, comprising one or more of the following N-glycosylated optimized amino acid sequence(s):

D/E-X-N-Z-S/T, (optimized consensus sequence)

wherein X and Z may be any natural amino acid except Pro, and wherein at least one of said N-glycosylated partial amino acid sequence(s) is introduced.

It was surprisingly found that the introduction of specific partial amino acid sequence(s)

(optimized consensus sequence(s)) into proteins leads to proteins that are efficiently N-glycosylated by the oligosaccharyl transferase (OST, OTase) from Campylobacter spp., preferably C. jejuni, in these introduced positions.

The term “partial amino acid sequence(s)” as it is used in the context of the present invention will also be referred to as “optimized consensus sequence(s)” or “consensus sequence(s)”. The optimized consensus sequence is N-glycosylated by the oligosaccharyl transferase (OST, OTase) from Campylobacter spp., preferably C. jejuni, much more efficiently than the regular consensus sequence “N-X-S/T” known in the prior art.

In general, the term “recombinant N-glycosylated protein” refers to any heterologous poly- or oligopeptide produced in a host cell that does not naturally comprise the nucleic acid encoding said protein. In the context of the present invention, this term refers to a protein produced recombinantly in any host cell, e.g. an eukaryotic or prokaryotic host cell, preferably a procaryotic host cell, e.g. Escherichia ssp., Campylobacter ssp., Salmonella ssp., Shigella ssp., Helicobacter ssp., Pseudomonas ssp., Bacillus ssp., more preferably Escherichia coli, Campylobacter jejuni, Salmonella typhimurium etc., wherein the nucleic acid encoding said protein has been introduced into said host cell and wherein the encoded protein is N-glycosylated by the OTase from Campylobacter spp., preferably C. jejuni, said transferase enzyme naturally occurring in or being introduced recombinantly into said host cell.

In accordance with the internationally accepted one letter code for amino acids the abbreviations D, E, N, S and T denote aspartic acid, glutamic acid, asparagine, serine, and threonine, respectively. Proteins according to the invention differ from natural or prior art proteins in that one or more of the optimized consensus sequence(s) D/E-X-N-Z-S/T is/are introduced and N-glycosylated. Hence, the proteins of the present invention differ from the naturally occurring C. jejuni proteins which also contain the optimized consensus sequence but do not comprise any additional (introduced) optimized consensus sequences.

The introduction of the optimized consensus sequence can be accomplished by the addition, deletion and/or substitution of one or more amino acids. The addition, deletion and/or substitution of one or more amino acids for the purpose of introducing the optimized consensus sequence can be accomplished by chemical synthetic strategies well known to those skilled in the art such as solid phase-assisted chemical peptide synthesis. Alternatively, and preferred for larger polypeptides, the proteins of the present invention can be prepared by standard recombinant techniques.

The proteins of the present invention have the advantage that they may be produced with high efficiency and in any procaryotic host comprising a functional pgl operon from Campylobacter spp., preferably C. jejuni. Preferred alternative OTases from Campylobacter spp. for practicing the aspects and embodiments of the present invention are Campylobacter coli and Campylobacter lari (see Szymanski, C M. and Wren, B. W. (2005). Protein glycosylation in bacterial mucosal pathogens. Nat. Rev. Microbiol. 3:225-237). The functional pgl operon may be present naturally when said procaryotic host is Campylobacter spp., preferably C. jejuni. However, as demonstrated before in the art and mentioned above, the pgl operon can be transferred into cells and remain functional in said new cellular environment.

The term “functional pgl operon from Campylobacter spp., preferably C. jejuni” is meant to refer to the cluster of nucleic acids encoding the functional oligosaccharyl transferase (OTase) of Campylobacter spp., preferably C. jejuni, and one or more specific glycosyltransferases capable of assembling an oligosaccharide on a lipid carrier, and wherein said oligosaccharide can be transferred from the lipid carrier to the target protein having one or more optimized amino acid sequence(s): D/E-X N-Z-S/T by the OTase. It to be understood that the term “functional pgl operon from Campylobacter spp., preferably C. jejuni′ in the context of this invention does not necessarily refer to an operon as a singular transcriptional unit. The term merely requires the presence of the functional components for N-glycosylation of the recombinant protein in one host cell. These components may be transcribed as one or more separate mRNAs and may be regulated together or separately. For example, the term also encompasses functional components positioned in genomic DNA and plasmid(s) in one host cell. For the purpose of efficiency, it is preferred that all components of the functional pgl operon are regulated and expressed simultaneously.

It is important to realize that only the functional oligosaccharyl transferase (OTase) should originate from Campylobacter spp., preferably C. jejuni, and that the one or more specific glycosyltransferases capable of assembling an oligosaccharide on a lipid carrier may originate from the host cell or be introduced recombinantly into said host cell, the only functional limitation being that the oligosaccharide assembled by said glycosyltransferases can be transferred from the lipid carrier to the target protein having one or more optimized consensus sequences by the OTase. Hence, the selection of the host cell comprising specific glycosyltransferases naturally and/or incapacitating specific glycosyltransferases naturally present in said host as well as the introduction of heterologous specific glycosyltransferases will enable those skilled in the art to vary the N-glycans bound to the optimized N-glycosylation consensus site in the proteins of the present invention.

As a result of the above, the present invention provides for the individual design of N-glycan-patterns on the proteins of the present invention. The proteins can therefore be individualized in their N-glycan pattern to suit biological, pharmaceutical and purification needs.

In a preferred embodiment, the proteins of the present invention may comprise one but also more than one, preferably at least two, preferably at least 3, more preferably at least 5 of said N-glycosylated optimized amino acid sequences.

The presence of one or more N-glycosylated optimized amino acid sequence(s) in the proteins of the present invention can be of advantage for increasing their antigenicity, increasing their stability, affecting their biological activity, prolonging their biological half-life and/or simplifying their purification.

The optimized consensus sequence may include any amino acid except proline in position(s) X and Z. The term “any amino acids” is meant to encompass common and rare natural amino acids as well as synthetic amino acid derivatives and analogs that will still allow the optimized consensus sequence to be N-glycosylated by the OTase. Naturally occurring common and rare amino acids are preferred for X and Z. X and Z may be the same or different.

It is noted that X and Z may differ for each optimized consensus sequence in a protein according to the present invention.

The N-glycan bound to the optimized consensus sequence will be determined by the specific glycosyltransferases and their interaction when assembling the oligosaccharide on a lipid carrier for transfer by the OTase. Those skilled in the art can design the N-glycan by varying the type(s) and amount of the specific glycosyltransferases present in the desired host cell.

N-glycans are defined herein as mono-, oligo- or polysaccharides of variable compositions that are linked to an s-amide nitrogen of an asparagine residue in a protein via an N-glycosidic linkage. Preferably, the N-glycans transferred by the OTase are assembled on an undecaprenol-pyrophosphate lipid-anchor that is present in the cytoplasmic membrane of gram-negative or positive bacteria. They are involved in the synthesis of O antigen, O polysaccharide and peptidoglycan (Bugg, T. D., and Brandish, P. E. (1994). From peptidoglycan to glycoproteins: common features of lipid-linked oligosaccharide biosynthesis. FEMS Microbiol Lett 119, 255-262; Valvano, M. A. (2003). Export of O-specific lipopolysaccharide. Front Biosci 8, S452-471).

In a preferred embodiment, the recombinant protein of the present invention comprises one or more N-glycans selected from the group of N-glycans from Campylobacter spp., preferably C. jejuni, the N-glycans derived from oligo- and polysaccharides transferred to O antigen forming O polysaccharide in Gram-negative bacteria or capsular polysaccharides from Gram-positive bacteria, preferably: P. aeruginosa 09, 011; E. coli 07, 09, 016, 0157 and Shigella dysenteriae OI and engineered variants thereof obtained by inserting or deleting glycosyltransferases and epimerases affecting the polysaccharide structure.

In a further preferred embodiment, the recombinant protein of the present invention comprises two or more different N-glycans.

For example, different N-glycans on the same protein can prepared by controlling the timing of the expression of specific glycosyltransferases using early or late promoters or introducing factors for starting, silencing, enhancing and/or reducing the promoter activity of individual specific glycosyltransferases. Suitable promoters and factors governing their activity are routinely available to those in the art.

There is no limitation on the origin of the recombinant protein of the invention. Preferably said protein is derived from mammalian, bacterial, viral, fungal or plant proteins. More preferably, the protein is derived from mammalian, most preferably human proteins. For preparing antigenic recombinant proteins according to the invention, preferably for use as active components in vaccines, it is preferred that the recombinant protein is derived from a bacterial, viral or fungal protein.

In a further preferred embodiment, the present invention provides for recombinant proteins wherein either the protein and/or the N-glycan(s) is (are) therapeutically and/or prophylactically active. The introduction of at least one optimized and N-glycosylated consensus sequence can modify or even introduce therapeutic and/or prophylactic activity in a protein. In a more preferred embodiment, it is the protein and/or the N-glycan(s) that is (are) immunogenically active. In this case, the introduced N-glycosylation(s) may have a modifying effect on the proteins biological activity and/or introduce new antigenic sites and/or may mask the protein to evade degrading steps and/or increase the half-life.

The recombinant proteins of the present invention can be efficiently targeted to the outer membrane and/or surface of host cells, preferably bacteria, more preferably gram-negative bacteria. For assisting the surface display and/or outer membrane localization, it is preferred that the recombinant protein of the invention further comprise at least one polypeptide sequence capable of targeting said recombinant protein to the outer membrane and/or cell surface of a bacterium, preferably a gram-negative bacterium.

In a preferred embodiment, the recombinant protein of the invention is one, wherein said targeting polypeptide sequence is selected from the group consisting of type II signal peptides (Paetzel, M., Karla, A., Strynadka, N.C., and Dalbey, R. E. 2002. Signal peptidases. Chem Rev 102: 4549-4580.) or outer membrane proteins (reviewed in Wemerus, H., and Stahl, S. 2004. Biotechnological applications for surface-engineered bacteria. Biotechnol Appl Biochem 40: 209-228.), preferably selected from the group consisting of the full length protein or the signal peptides of OmpHI from C. jejuni, JIpA from C. jejuni, outer membrane proteins from E. coli, preferably OmpS, OmpC, OmpA, OprF, PhoE, LamB, Lpp′OmpA (a fusion protein for surface display technology, see Francisco, JAi Earhart, C. F., and Georgiou, G. 1992. Transport and anchoring of beta-lactamase to the external surface of Escherichia coli. Proc Natl Acad Sci USA 89: 2713-2717.), and the Inp protein from Pseudomonas aeruginosa.

In a different aspect, the present invention relates to a nucleic acid encoding a recombinant protein according to the invention. Preferably, said nucleic acid is a mRNA, a DNA or a PNA, more preferably a mRNA or a DNA, most preferably a DNA. The nucleic acid may comprise the sequence coding for said protein and, in addition, other sequences such as regulatory sequences, e.g. promoters, enhancers, stop codons, start codons and genes required to regulate the expression of the recombinant protein via the mentioned regulatory sequences, etc. The term “nucleic acid encoding a recombinant protein according to the invention” is directed to a nucleic acid comprising said coding sequence and optionally any further nucleic acid sequences regardless of the sequence information as long as the nucleic acid is capable of producing the recombinant protein of the invention in a host cell containing a functional #g7 operon from Campylobacter spp., preferably C. jejuni. More preferably, the present invention provides isolated and purified nucleic acids operably linked to a promoter, preferably linked to a promoter selected from the group consisting of known inducible and constitutive prokaryotic promoters, more preferably the tetracycline promoter, the arabinose promoter, the salicylate promoter, lac-, trc-, and tac promotors (Baneyx, F. (1999). Recombinant protein expression in Escherichia coli. Curr Opin Biotechnol 10, 411-421; Billman-Jacobe, H. (1996). Expression in bacteria other than Escherichia coli. Curr Opin Biotechnol 7, 500-504.). Said operably linked nucleic acids can be used for, e.g. vaccination.

Furthermore, another aspect of the present invention relates to a host cell comprising a nucleic acid and/or a vector according to the present invention. The type of host cell is not limiting as long as it accommodates a functional pgl operon from C. jejuni and one or more nucleic acids coding for recombinant target protein(s) of the present invention. Preferred host cells are prokaryotic host cells, more preferably bacteria, most preferably those selected from the group consisting of Escherichia ssp., Campylobacter ssp., Salmonella ssp., Shigella ssp., Helicobacter ssp., Pseudomonas ssp., Bacillus ssp., preferably Escherichia coli, more preferably E. coli strains TopIO, W3110, CLM24, BL21, SCM6 and SCM7 (Feldman et al., (2005). Engineering N-linked protein glycosylation with diverse O antigen lipopolysaccharide structures in Escherichia coli. Proc. Natl. Acad. Sci. USA 102, 3016-3021; Alaimo, C, Catrein, I., Morf, L., Marolda, C. L., Callewaert, N., Valvano, M. A., Feldman, M. F., Aebi, M. (2006). Two distinct but interchangeable mechanisms for flipping of lipid-linked oligosaccharides. EMBO Journal 25, 967-976) and S. enterica strains SL3261 {Salmonella enterica sv. Typhimurium LT2 (delta) aroA, see Hoiseth, S. K., and Stocker, B. A. 1981, Aromatic-dependent Salmonella typhimurium are non-virulent and effective as live vaccines. Nature

291:238-239), SL3749 {Salmonella enterica sv. Typhimurium LT2 waaL, see Kaniuk et al., J. Biol. Chem. 279: 36470-36480) and SL3261 ΔwaaL.

In a more preferred embodiment, the host cell according to the invention is one that is useful for the targeting to the outer membrane and/or surface display of recombinant proteins according to the invention, preferably one, wherein said host cell is a recombinant gram-negative bacterium having:
i) a genotype comprising nucleotide sequences encoding for
a) at least one natural or recombinant specific glycosyltransferase for the assembly of an oligosaccharide on a lipid carrier,
b) at least one natural or recombinant prokaryotic oligosaccharyl transferase (OTase) from Campylobacter spp., preferably C. jejuni,
c) at least one recombinant protein according to the invention, preferably a protein further comprising a targeting polypeptide, and ii) a phenotype comprising a recombinant N-glycosylated protein according to the invention that is located in and/or on the outer membrane of the gram-negative bacterium.

The host cell for the above embodiment is preferably selected from the group consisting of Escherichia ssp., Campylobacter ssp., Shigella ssp, Helicobacter ssp. and Pseudomonas ssp., Salmonella ssp., preferably E. coli, more preferably E. coli strains Top 10, W3110, CLM24, BL21, SCM6 and SCM7, and S. enterica strains SL3261, SL3749 and SL326i6waaL (see Hoiseth, S. K., and Stocker, B. A. 1981. Aromatic-dependent Salmonella typhimurium are non-virulent and effective as live vaccines. Nature 291: 238-239), SL3749 (Kaniuk, N. A., Vinogradov, E., and Whitfield, C. 2004. Investigation of the structural requirements in the lipopolysaccharide core acceptor for ligation of 0 antigens in the genus Salmonella: WaaL “ligase” is not the sole determinant of acceptor specificity. J Biol Chem 279: 36470-36480).

Because preferred proteins of the present invention may have a therapeutic or prophylactic activity by themselves and/or due to the introduced N-glycosylation sites, they can be used for the preparation of a medicament. The type of protein for practicing the invention is not limited and, therefore, proteins of the invention such as EPO, IFN-alpha, TNFalpha, IgG, IgM, IgA, interleukins, cytokines, viral and bacterial proteins for vaccination like C. jejuni proteins such as HisJ (Cj0734c), AcrA (Cj0367c), OmpHI (Cj0982c), Diphteria toxin (CRM 197), Cholera toxin, P. aeruginosa exoprotein, to name just a few, and having introduced therein the optimized N-glycosylated consensus sequence are useful for preparing a medicament (Wyszynska, A., Raczko, A., Lis, M., and Jagusztyn-Krynicka, E. K. (2004). Oral immunization of chickens with avirulent Salmonella vaccine strain carrying C. jejuni 72Dz/92 cjaA gene elicits specific humoral immune response associated with protection against challenge with wild-type Campylobacter. Vaccine 22, 1379-1389).

In addition, the nucleic acids and/or vectors according to the invention are also useful for the preparation of a medicament, preferably for use in gene therapy.

Moreover, a host cell according to the invention, preferably one that has a phenotype comprising an N-glycosylated recombinant protein of the invention that is located in and/or on the outer membrane of a bacterium, preferably a gram-negative bacterium, more preferably one of the above-listed gram-negative bacteria, is particularly useful for the preparation of a medicament.

More preferably, a protein of the invention is used for the preparation of a medicament for the therapeutic and/or prophylactic vaccination of a subject in need thereof.

In a more preferred embodiment, the present invention relates to the use of a nucleic acid and/or a vector according to the invention for the preparation of a medicament for the therapeutic and/or prophylactic vaccination of a subject in need thereof, preferably by gene therapy.

The host cells of the invention displaying said N-glycosylated recombinant proteins are particularly useful for preparing vaccines, because the displayed N-glycosylated proteins are abundantly present on the host cell s surface and well accessible by immune cells, in particular their hydrophilic N-glycans, and because the host cells have the added effect of an adjuvant, that, if alive, may even replicate to some extent and amplify its vaccination effects.

Preferably, the host cell for practicing the medical aspects of this invention is an attenuated or killed host cell.

Another advantage of the use of the inventive host cells for preparing medicaments, preferably vaccines, is that they induce IgA antibodies due to the cellular component.

Preferably, said host cells are used according to the invention for inducing IgA antibodies in an animal, preferably a mammal, a rodent, ovine, equine, canine, bovine or a human. It is preferred that said subject in need of vaccination is avian, mammalian or fish, preferably mammalian, more preferably a mammal selected from the group consisting of cattle, sheep, equines, dogs, cats, and humans, most preferably humans. Fowls are also preferred.

A further aspect of the present invention relates to a pharmaceutical composition, comprising at least one protein, at least one nucleic acid, a least one vector and/or at least one host cell according to the invention. The preparation of medicaments comprising proteins or host cells, preferably attenuated or killed host cells, and the preparation of medicaments comprising nucleic acids and/or vectors for gene therapy are well known in the art. The preparation scheme for the final pharmaceutical composition and the mode and details of its administration will depend on the protein, the host cell, the nucleic acid and/or the vector employed.

In a preferred embodiment, the pharmaceutical composition of the invention comprises a pharmaceutically acceptable excipient, diluent and/or adjuvant.

The present invention provides for a pharmaceutical composition comprising at least one of the following, (i) a recombinant protein, a host cell, a nucleic acid and/or a recombinant vector being/encoding/expressing a recombinant protein according to the present invention, and (ii) a pharmaceutically acceptable excipient, diluent and/or adjuvant.

Suitable excipients, diluents and/or adjuvants are well-known in the art. An excipient or diluent may be a solid, semi-solid or liquid material which may serve as a vehicle or medium for the active ingredient. One of ordinary skill in the art in the field of preparing compositions can readily select the proper form and mode of administration depending upon the particular characteristics of the product selected, the disease or condition to be treated, the stage of the disease or condition, and other relevant circumstances (Remington s Pharmaceutical Sciences, Mack Publishing Co. (1990)). The proportion and nature of the pharmaceutically acceptable diluent or excipient are determined by the solubility and chemical properties of the pharmaceutically active compound selected, the chosen route of administration, and standard pharmaceutical practice. The pharmaceutical preparation may be adapted for oral, parenteral or topical use and may be administered to the patient in the form of tablets, capsules, suppositories, solution, suspensions, or the like. The pharmaceutically active compounds of the present invention, while effective themselves, can be formulated and administered in the form of their pharmaceutically acceptable salts, such as acid addition salts or base addition salts, for purposes of stability, convenience of crystallization, increased solubility, and the like.

A further aspect of the present invention is directed to a method for producing N-linked glycosylated proteins, comprising the steps of:
a) providing a recombinant organism, preferably a prokaryotic organism, comprising nucleic acids coding for
i) a functional pgl operon from Campylobacter spp., preferably C. jejuni, and ii) at least one recombinant target protein comprising one or more of the following N-glycosylated optimized amino acid consensus sequence(s):

D/E-X-N-Z-S/T,

wherein X and Z may be any natural amino acid except Pro, and wherein at least one of said N-glycosylated optimized amino acid consensus sequence(s) is introduced, and
b) culturing the recombinant organism in a manner suitable for the production and N-glycosylation of the target protein(s).

Preferably, the target protein is one of the above described recombinant proteins according to the invention.

In a preferred method of the invention, the functional pgl operon from Campylobacter spp., preferably C. jejuni, comprises nucleic acids coding for
i) recombinant OTase from Campylobacter spp., preferably C. jejuni, and
ii) recombinant and/or natural specific glycosyltransferases from Campylobacter spp., preferably C. jejuni, and/or
iii) recombinant and/or natural specific glycosyltransferases from species other than

Campylobacter spp.,

for the assembly of an oligosaccharide on a lipid carrier to be transferred to the target protein by the OTase.

Moreover, in a preferred embodiment the present invention relates to a method for preparing a host cell according to the invention comprising the steps of:
i) providing a gram-negative bacterium,
ii) introducing into said bacterium at least one nucleotide sequence encoding for
a) at least one recombinant specific glycosyltransferase for the assembly of an oligosaccharide on a lipid carrier, and/or b) at least one recombinant oligosaccharyl transferase (OTase) from Campylobacter spp., preferably C. jejuni, and/or
c) at least one recombinant protein comprising one or more of the following N-glycosylated optimized amino acid consensus sequence(s):

D/E-X-N-Z-S/T,

wherein X and Z may be any natural amino acid except Pro, and wherein at least one of said N-glycosylated optimized amino acid consensus sequence(s) is introduced, and
iii) culturing said bacterium until at least one recombinant N-glycosylated protein coded by the nucleotide sequence of c) is located in and/or on the outer membrane of the gram-negative bacterium.

For practicing the preferred methods above, the recombinant procaryotic organism or host cell is preferably selected from the group of bacteria consisting of Escherichia ssp., Campylobacter ssp., Salmonella ssp., Shigella ssp., Helicobacter ssp., Pseudomonas ssp., Bacillus ssp., preferably Escherichia coli, preferably E. coli strains Top 10, W3110, W31 10A waaL, BL21, SCM6 and SCM7, and S. enterica strains SL3261, SL3749 and SL3261 ΔwaaL.

Another preferred method for producing, isolating and/or purifying a recombinant protein according to the invention comprises the steps of:
a) culturing a host cell,
b) removing the outer membrane of said recombinant gram-negative bacterium; and
c) recovering said recombinant protein.

Exemplary methods for removing the outer membrane of a cell, preferably a prokaryotic cell, more preferably a gram-negative bacterial cell, are suitable enzymatic treatment methods, osmotic shock detergent solubilisation and the French press method.

Most preferred, the present invention relates to a method, wherein recombinant or natural specific glycosyltransferases from species other than Campylobacter spp., preferably C. jejuni, are selected from the group of glycosyltransferases and epimerases originating from bacteria, archea, and/or eukaryota that can be functionally expressed in said host cell.

### Bioconjugate Vaccines

An embodiment of the invention involves novel bioconjugate vaccines. A further embodiment of the invention involves a novel approach for producing such bioconjugate vaccines that uses recombinant bacterial cells that directly produce immunogenic or antigenic bioconjugates. In one embodiment, bioconjugate vaccines can be used to treat or prevent bacterial diseases, such as diarrhea, nosocomial infections and meningitis. In further embodiments, biooconjugate vaccines may have therapeutic and/or prophylactic potential for cancer or other diseases.

Conjugate vaccines can be administered to children to protect against bacterial infections and can provide a long lasting immune response to adults. Constructs of the invention have been found to generate an IgG response in animals. It has been found that an IgG response to a Shigella O-specific polysaccharide-protein conjugate vaccine in humans correlates with immune protection in humans. (Passwell, J. H. et al, “Safety and Immunogenicity of Improved Shigella O-Specific Polysaccharide-Protein Conjugate Vaccines in Adults in Israel” Infection and Immunity, 69(3):1351-1357 (March 2001).) It is believed that the polysaccharide (i.e. sugar residue) triggers a short-term immune response that is sugar-specific. Indeed, the human immune system generates a strong response to specific polysaccharide surface structures of bacteria, such as O-antigens and capsular polysaccharides. However, since the immune response to polysaccharides is IgM dependent, the immune system develops no memory. The protein carrier that carries the polysaccharide triggers an IgG response that is T-cell dependent and that provides long lasting protection since the immune system develops memory.

A typical vaccination dosage for humans is about 1 to 25 μg, preferably about 1 μg to about IO μg, most preferably about 10 μg. Optionally, a vaccine, such as a bioconjugate vaccine of the present invention, includes an adjuvant.

Synthesized complexes of polysaccharides (i.e., sugar residues) and proteins (i.e., protein carriers) can be used as conjugate vaccines to protect against a number of bacterial infections. In one aspect, the instant invention is directed to a novel bioengineering approach for producing immunogenic conjugate vaccines that provide advantages over classical chemical conjugation methods. In one embodiment, the approach involves in vivo production of glycoproteins in bacterial cells, for example, Gram-negative cells such as E. coli.

The biosynthesis of different polysaccharides is conserved in bacterial cells. The polysaccharides are assembled on carrier lipids from common precursors (activated sugar nucleotides) at the cytoplasmic membrane by different glycosyltransferases with defined specificity. Lipopolysaccharides (LPS) are provided in gram-negative bacteria only, e.g. Shigella spp., Pseudomonas spp. and E. coli (ExPEC, EHEC).

The synthesis of lipopolysaccharides (LPS) starts with the addition of a monosaccharide to the carrier lipid undecaprenyl phosphate at the cytoplasmic side of the membrane. The antigen is built up by sequential addition of monosaccharides from activated sugar nucleotides by different glycosyltransferases and the lipid-linked polysaccharide is flipped through the membrane by a flippase. The antigen-repeating unit is polymerized by an enzymatic reaction. The polysaccharide is then transferred to the Lipid A by the Ligase WaaL forming the LPS that is exported to the surface, whereas the capsular polysaccharide is released from the carrier lipid after polymerization and exported to the surface. The biosynthetic pathway of these polysaccharides enables the production of LPS bioconjugates in vivo, capturing the polysaccharides in the periplasm to a protein carrier. Bioconjugates, such as LPS bioconjugates, are preferred in the present invention.

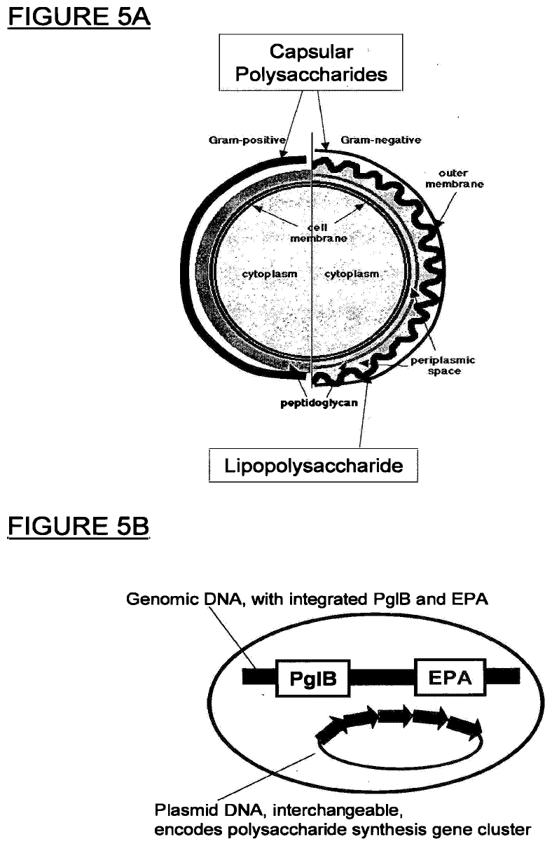

As shown in FIG. 5A, both Gram-positive and Gram-negative bacteria have a cell membrane that is surrounded by capsular polysaccharides. Gram-negative bacteria additionally have an outer membrane over the cell membrane, with a periplasmic space separating the two membranes. In addition, they contain lipopolysacharides at the surface. When Gram-negative bacteria, such as E. coli, is used to produce a conjugate vaccine of the present invention, the glycoprotein used in the conjugate vaccine is assembled in the periplasmic space.

Conjugate vaccines have been successfully used to protect against bacterial infections. The conjugation of an antigenic polysaccharide to a protein carrier is required for protective memory response, as polysaccharides are T-cell independent antigens. Polysaccharides have been conjugated to protein carriers by different chemical methods, using activation reactive groups in the polysaccharide as well as the protein carrier.

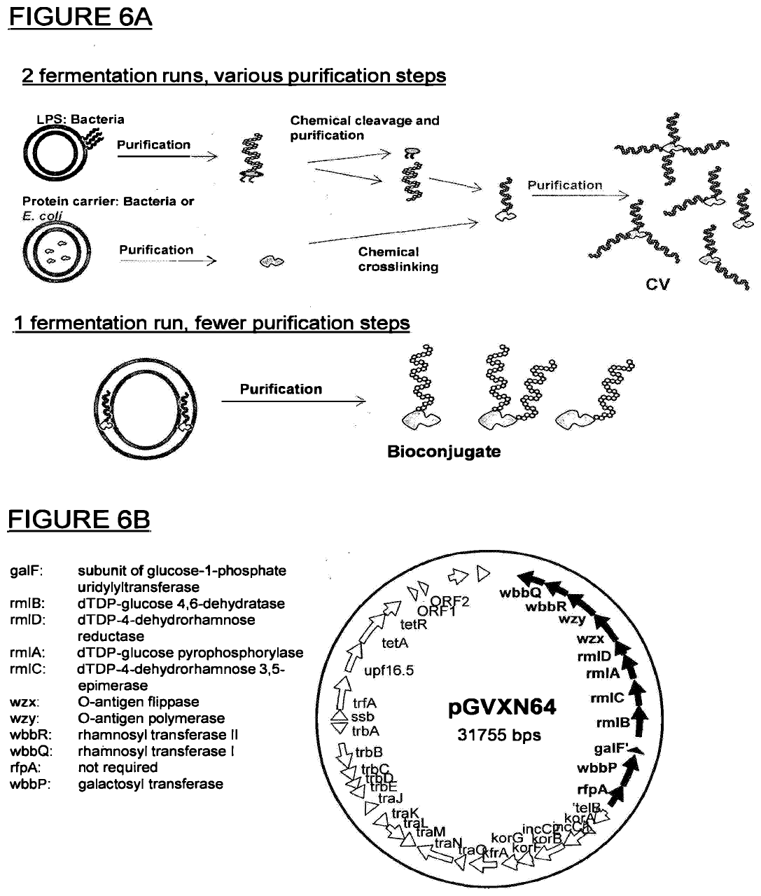

FIG. 6A shows the production process of conjugate vaccines using technology of the invention compared to the currently used process. Currently, conjugate vaccines are produced using two fermentation runs and after several purification and chemical cleavage steps, as schematically shown in the top panel. This current approach has a number of problems. First, large scale cultivation of pathogenic organisms is required. Second, the conjugation approach is dependent on the polysaccharide. Third, the approach has low reproducibility. Fourth, the approach has low homogeneity due to unspecific conjugation. Fifth, the approach also has low purity due to excess of polysaccharide in conjugation. Finally, the current approach provides yields of less than 20%.

As shown in the bottom panel of FIG. 6A, in an embodiment, the innovative technology of the invention can be used to develop conjugate vaccines (e.g., bioconjugate vaccines) completely in vivo with non-pathogenic cells, avoiding chemical reactions and providing high purity after a few purification steps. This novel method also allows for the production of bioconjugate vaccines that are not feasible using current methods. Moreover, the conjugation and purification process is independent of the polysaccharide antigen that is used. As a result, bioconjugate vaccines can be engineered faster using novel glycan structures. The increased homogeneity of resulting conjugates and the improved reproducibility (i.e., no batch to batch variability) of such conjugates makes this a highly attractive process from quality control and regulatory perspectives. In addition, the novel method provides good yield (30-60 mg/L and up to 200 mg/L).

The present invention is directed to a novel conjugation process involving engineering bacterial cells to produce the final bioconjugate vaccines. One embodiment of the invention allows the production of bioconjugate vaccines in vivo, circumventing the chemical conjugation and therefore simplifying the production process. The technology includes a novel genetic/enzymatic mechanism for the in vivo synthesis of novel bioconjugates consisting of protein-linked saccharides.

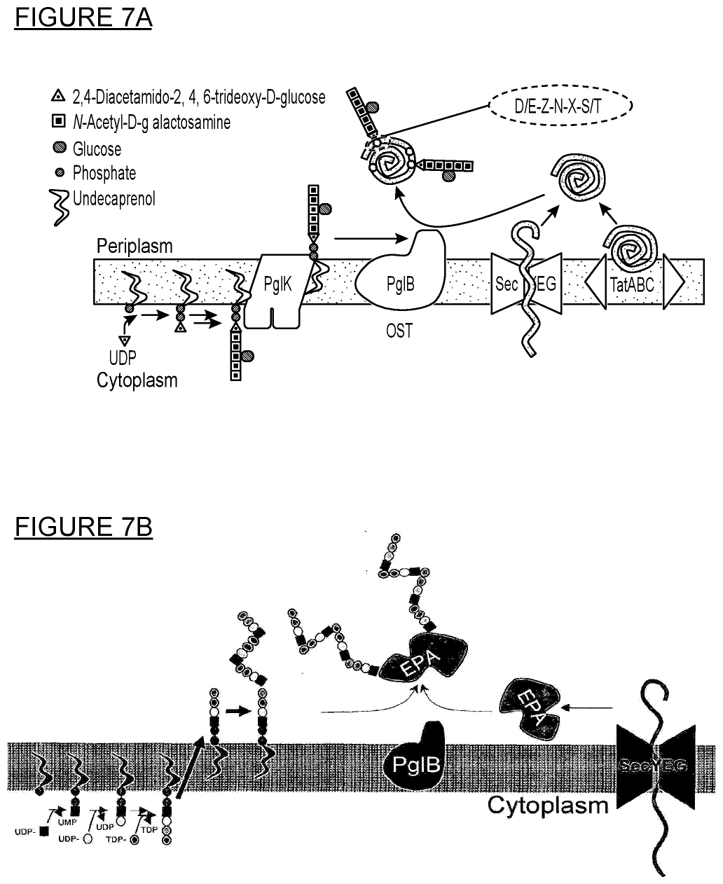

The basis of one aspect of the invention includes the discovery that Campylobacter jejuni contains a general N-linked protein glycosylation system, an unusual feature for prokaryotic organisms. Various proteins of C. jejuni have been shown to be modified by a heptasaccharide. This heptasaccharide is assembled on undecaprenyl pyrophosphate, the carrier lipid, at the cytoplasmic side of the inner membrane by the stepwise addition of nucleotide activated monosaccharides catalyzed by specific glycosyltransferases. The lipid-linked oligosaccharide then flip-flops (diffuses transversely) into the periplasmic space by a flipppase, e.g., PgIK. In the final step of N-linked protein glycosylation, the oligosaccharyltransferase (e.g., PglB) catalyzes the transfer of the oligosaccharide from the carrier lipid to Asn residues within the consensus sequence Asp/Glu-Xaa-Asn-Zaa-Ser/Thr (i.e., D/E-X N-Z-S/T), where the Xaa and Zaa can be any amino acid except Pro (FIG. 7A). We have successfully transferred the glycosylation cluster for the heptasaccharide into E. coli and were able to produce N-linked glycoproteins of Campylobacter.

We have been able to demonstrate that PglB does not have a strict specificity for the lipid-linked sugar substrate. The antigenic polysaccharides assembled on undecaprenyl pyrophosphate are captured by PglB in the periplasm and transferred to a protein carrier (Feldman, 2005; Wacker, M., et al., Substrate specificity of bacterial oligosaccharyltransferase suggests a common transfer mechanism for the bacterial and eukaryotic systems. Proc Natl Acad Sci USA, 2006. 103(18): p. 7088-93.) The enzyme will also transfer a diverse array of undecaprenyl pyrophosphate (UPP) linked oligosaccharides if they contain an N-acetylated hexosamine at the reducing terminus. The nucleotide sequence for pglB is provided at SEQ. ID NO. 1, whereas the amino acid sequence for PglB is provided at SEQ. ID. NO. 2.

FIGS. 7 A and 7B show schematics of the protein glycosylation pathway (i.e., N-glycosylation system) of the present invention. In an embodiment, the protein glycosylation pathway of C. jejuni (e.g., including pgl operon) can be introduced into E. coli. In FIG. 7A, an oligosaccharide, specifically a heptasaccharide made of five N-acetyl-D-galactosamine units, one glucose unit and one 2,4-diacetamido-2,4,6-trideoxy-D-glucose unit, is assembled onto a lipid carrier, undecaprenylpyrophosphate (UDP), using glycosyltransferases (e.g., pglA, pglC, pglH, J; I) at the cytoplasmic side of the inner membrane and is transferred to the periplasmic space by way of a flippase called PgIK. Separately, a carrier protein depicted as a spiral and containing consensus sequence D/E-X-N-Z-S/T (i.e., Asp/Glu-Xaa-Asn-Zaa-Ser/Thr) is translated in the cytoplasm and is secreted into the periplasmic space. In the final step, an oligosaccharyl transferase (OST or OTase) (e.g., PglB) transfers the heptasaccharide to Asn residues within a consensus sequence of the carrier protein to produce a glycoprotein.

FIG. 7B also shows biosynthesis of a polysaccharide (i.e., an antigenic polysaccharide or antigen) by stepwise action of glycosyltransferases, and transfer of the O-antigen to the periplasm by way of flippase, followed by polymerization into a polysaccharide using a polymerase (e.g., wzy). Separately, a carrier protein, such as EPA, is produced and secreted into the periplasm. An oligosaccharyl transferase (OST or OTase), such as PglB, has relaxed substrate specificity and transfers the polysaccharide from a lipid carrier to Asn in the consensus sequence within EPA.

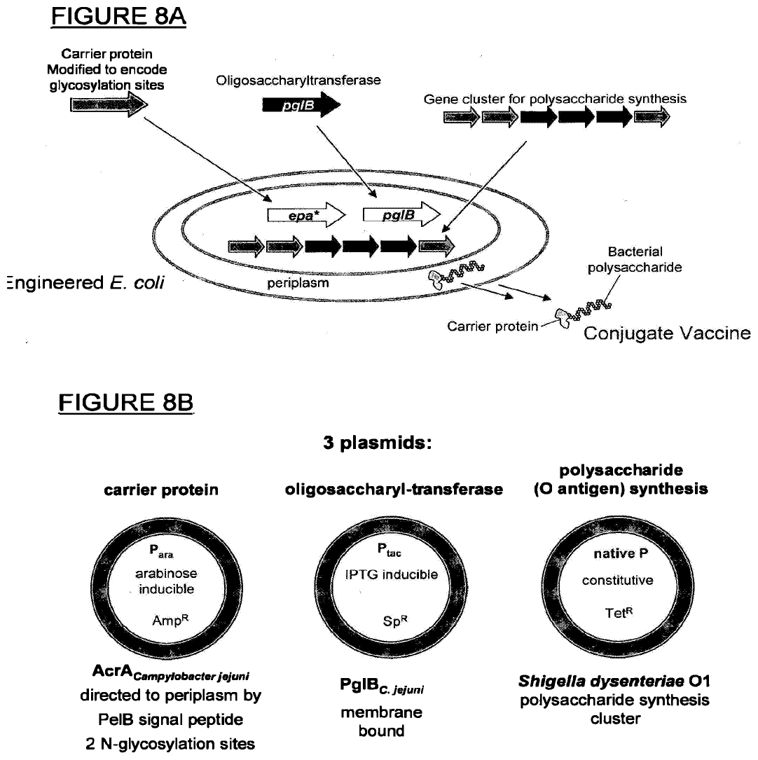

FIG. 8 A shows a schematic depicting an embodiment of the expression platform for bioconjugate production of the present invention. The technology of the invention is versatile in that various existing carrier proteins can be employed, so long as the carrier protein contains or is modified to contain the consensus sequence, as discussed earlier. In particular, FIG. 8A illustrates the construction of an expression host, such as an engineered E. coli bacterium in an embodiment of the invention. Such an E. coli contains the general components of a glycosylation system (i.e., an OST/OTase, e.g., PglB, and a protein carrier, e.g. EPA). Such components can be integrated into the genome of an E. coli strain. In addition, the Ligase WaaL as well as WecG are deleted. Additionally, specific components for polysaccharide antigen expression (i.e., a polysaccharide synthesis gene cluster containing, for example, glycosyl transferase, polymerase, flippase, and sugar biosynthesis enzymes) can be provided by the addition of an exchangeable plasmid. This construction allows for specific glycosylation of the protein carrier with a polysaccharide of choice in vivo.

In an embodiment of the expression system for a bacterial bioconjugate that is compatible with Good Manufacturing Practices (GMP), DNA encoding the inducible oligosaccharyltransferase and carrier protein can be stably integrated into a bacterial (e.g., E. coli) genome such that genes for antibiotic selection can be omitted. For example, as shown in FIG. 5B, PglB and EPA is integrated into genomic DNA, whereas plasmid DNA, which is interchangeable (i.e., exchangeable), encodes a polysaccharide synthesis gene cluster.

In another embodiment, FIG. 8B shows an expression system for a bacterial bioconjugate that includes three plasmids. A first plasmid codes for the carrier protein, e.g., AcrA from Campylobacter jejuni, which has two N-glycosylation sites and is directed to the periplasm by a PeIB signal peptide. A second plasmid codes for the OST/OTase, e.g., PglB from C. jejuni, which is membrane-bound. A third plasmid is a native plasmid that codes, e.g., for a polysaccharide (O antigen) synthesis cluster, such as that for Shigella dysenteriae 01.

In an embodiment, an expression plasmid for a bacterial O antigen, such as the Shigella dysenteriae OI antigen, can be constructed as in pGVXN64 shown in FIG. 6B. This plasmid encodes all enzymes necessary to synthesize the polysaccharides in the Shigella dysenteriae strain that make up the 01 serotype. These enzymes are listed in the left-hand column of FIG. 6B. Vector pGVXN64 expressing the Shigella dysenteriae OI antigen was constructed by digestion of pLARFRI (Vanbleu, E. et ah, “Genetic and physical map of the pLAFRI vector” DNA Seq. 15(3):225-227 (2004)) with EcoRI followed by insertion of an oligonucleotide cassette (5-AATTCTGC AGGATCCTCTAGAAGCTTGG (SEQ. ID NO. 3) and 5′-AATTCCAAGCTTCTAGAGGATCCTGCAG (SEQ. ID NO. 4). The BamHI fragment of pSDM7 (Fait, I. et al, “Construction of recombinant aroA salmonellae stably producing the Shigella Dysenteriae serotype 1 O-antigen and structural characterization of the Salmonella/Shigella hybrid LPS” Microb. Pathog. 20(1): 11-30 (1996)) containing the rfb and rfp cluster of Shigella dysenteriae OI was then cloned via the BamHI site into the oligonucleotide cassette containing pLAFRI. The complete nucleotide sequence encoding the Shigella dysenteriae OI antigen in the pGVXN64 plasmid is set forth as SEQ. ID NO.: 5 in the Sequence Listing provided below.

The host organism for an expression system of the invention can be, e.g., an Escherichia coli strain such as Escherichia coli W311 IOΔwaaL. The deletion of WaaL prevents the transfer of any polysaccharide to the lipid A core. The chromosomal copy of WaaL can also be replaced by PglB. The strain also contains mutation in wbbL, therefore it does not produce any E. coli 016 polysaccharide. To further increase the production of carrier lipid linked polysaccharide, wecG has been deleted to prevent the formation of ECA (Entero Common Antigen).

In one aspect, the instant invention is further directed to the development of bioconjugate vaccines, preferably LPS bioconjugate vaccines, against one or more Shigella species, which are invasive, gram-negative bacteria. Shigella species cause Shigellosis, a severe inflammation of the colon. There are 165 million cases in the world every year, with 70% of such cases being in children under 5 years of age. In developing countries, Shigellosis causes 1.1 million of deaths per year. This is a serious disease that is spread via the fecal-oral route and is highly transmissible. Potential groups that would benefit from immunization against Shigella species include, for example, children, travelers and people in refugee camps.

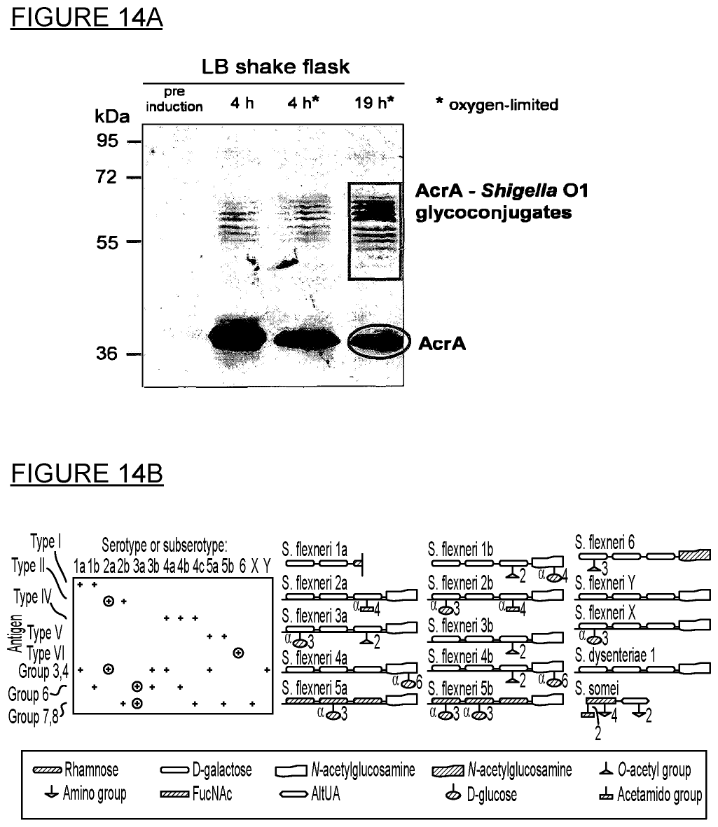

There are four different serogroups of Shigella, namely, S. dysenteriae, S. flexneri, S. sonnei and S. boydii. In embodiments of the present invention, immunogenic bioconjugates can be made against each of these different serogroups of Shigella. For example, FIG. 14B provides different serotypes of Shigella and the polysaccharide structure that defines their antigenicity (i.e., Shigella O-antigens).

In further embodiments of the present invention, immunogenic LPS bioconjugates could be made against other bacteria using the teachings in this specification, including bacteria: (1) that cause nosocomial infections, such as Pseudomonas aeruginosa; and (2) that cause urinary tract infection, such as Extraintestinal E. coli (ExPEC).

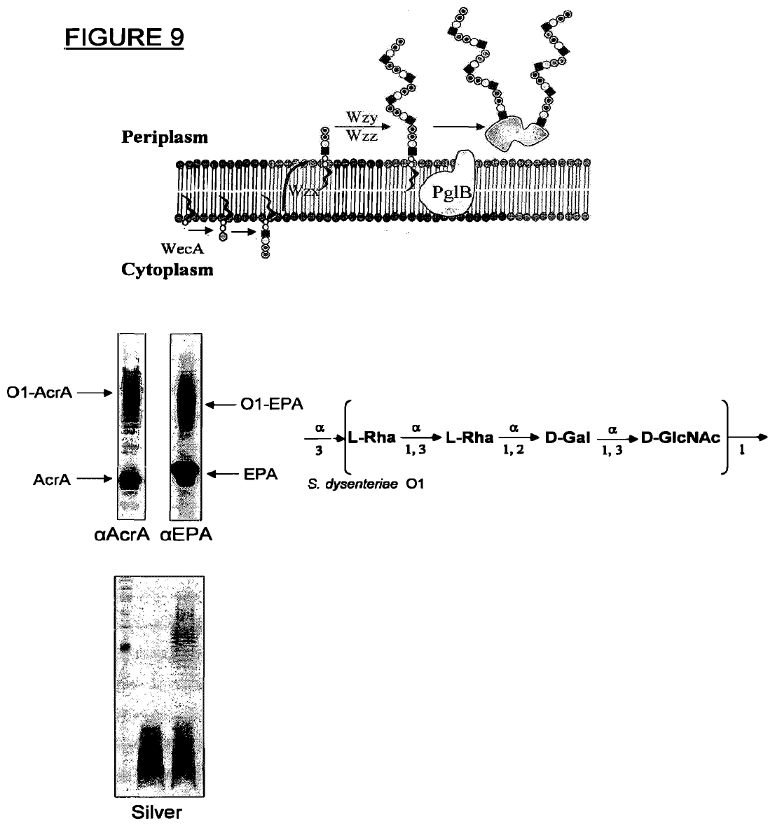

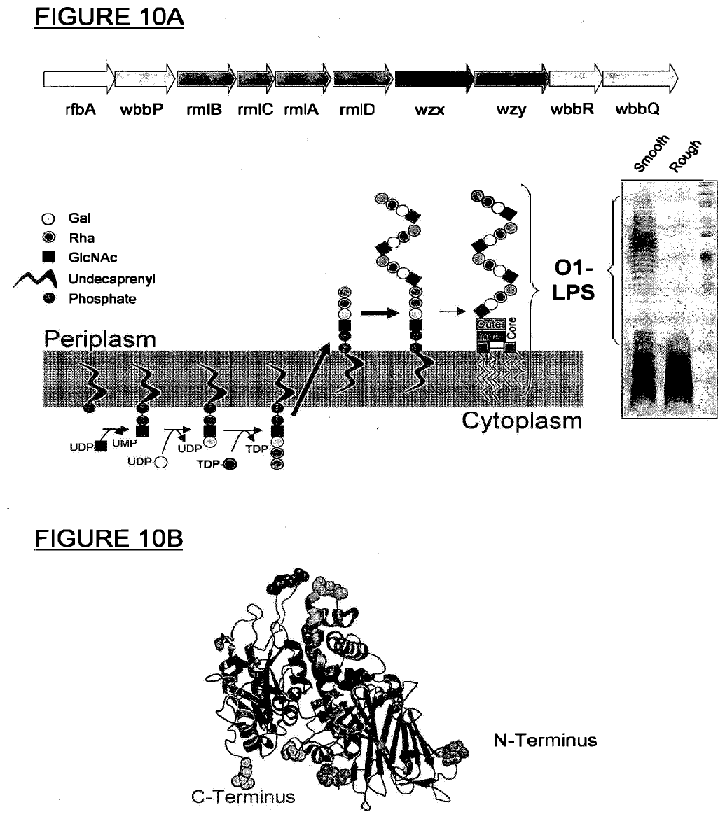

In an embodiment, the inventors have developed a Shigella dysenteriae OI LPS bioconjugate vaccine (also referred to as a S. dysenteriae bioconjugate), using genetically engineered E. coli with simple fermentation and purification methods. FIG. 9 shows production of Shigella bioconjugates. The top panel shows the synthesis of bioconjugates in E. coli. In an embodiment, the O-antigen repeating unit of S. dysenteriae OI is assembled on the carrier lipid undecaprenyl pyrophosphate (UPP), flipped to the periplasmic space and polymerized. The structure of S. dysenteriae OI is as follows and is also provided in the middle right of FIG. 9:

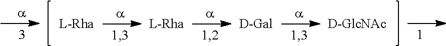

PglB transfers the activated polysaccharide to Asn residues of protein carriers, forming the Shigella bioconjugates. The protein carrier can be, for example, AcrA or a protein carrier that has been modified to contain the consensus sequence for protein glycosylation, i.e., D/E-X-N-Z-S/T, wherein X and Z can be any amino acid except proline (e.g., a modified Exotoxin Pseudomonas aeruginosa (EPA)). EPA has been used successfully in conjugate vaccines.

In an embodiment illustrated in FIG. 9, periplasmic proteins of E. coli cells expressing the modified EPA in the presence of PglB and the OI polysaccharide cluster were separated by SDS page and, after transfer to nitrocellulose, EPA was immunodetected with an antiserum that was raised against EPA (lane 2). In lane 1, periplasmic proteins of E. coli cells expressing the Campylobacter protein AcrA in the presence of PglB and the OI polysaccharide cluster were separated and immunodetected with an antiserum that was raised against AcrA. Both proteins were glycosylated with the 01-polysaccharide cluster. In the lowest panel of FIG. 9, the LPS from E. coli were separated by SDS-PAGE and visualized by Silver Staining. The left lane depicts the LPS extracted from a strain not expressing the WaaL, whereas the right lane shows the typical OI LPS pattern. Both strains are expressing the polysaccharide biosynthesis cluster of S. dysenteriae 01.

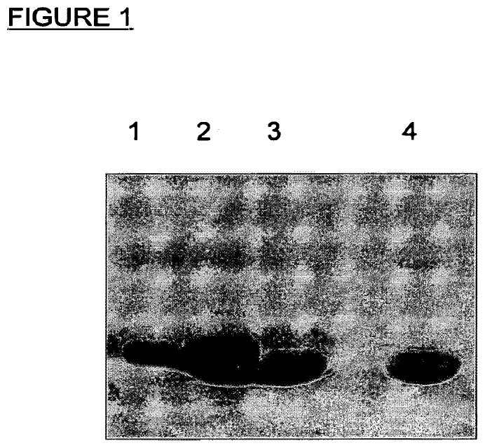

The production of a bacterial bioconjugate, such as a Shigella bioconjugate, is described in an embodiment in further detail with reference to FIGS. 1OA and 1OB. Prior to assembly of the bacterial bioconjugate using a bacterial system, such as E. coli, it is necessary to introduce into the bacterial system certain genetic sequences coding for the various enzymes and proteins to be used, as discussed earlier with reference to FIGS. 8A and 8B. For example, this includes an OST/Otase, preferably from C. jejuni (e.g., PglB), a protein carrier (e.g. EPA) and a gene cluster directed to antigenic polysaccharide synthesis (e.g., the gene cluster for S. dysenteriae OI polysaccharide synthesis).

FIG. 1OA shows Step 1 in the development of a bacterial bioconjugate, namely, the biosynthesis of a polysaccharide, such as the O-antigen of S. dysenteriae Serotype 01. In this step, the antigen is synthesized on the carrier lipid undecaprenyl pyrophosphate (UPP), and then transferred into the periplasm using a flippase. The antigen is polymerized by the polymerase Wzy and transferred to the lipid A core by the ligase WaaL. To transfer the polysaccharide to a protein carrier, the ligase is replaced by the oligosaccharyltransferase; PglB.

Step 2 in the production of a bacterial bioconjugate involves engineering a suitable protein carrier. Protein carriers that are useful preferably should have certain immunological and pharmacological features. From an immunological perspective, preferably, a protein carrier should: (1) have T-cell epitopes; (2) be capable of delivering an antigen to antigen presenting cells (APCs) in the immune system; (3) be potent and durable; and (4) be capable of generating an antigen-specific systemic IgG response. From a pharmacological perspective, preferably, a protein carrier should: (1) be non-toxic; and (2) be capable of delivering antigens efficiently across intact epithelial barriers. More preferably, in addition to these immunological and pharmacological features, a protein carrier suitable for the production of a bacterial bioconjugate should: (1) be easily secreted into the periplasmic space; and (2) be capable having antigen epitopes readily introduced as loops or linear sequences into it.

The inventors have found genetically detoxified Pseudomonas aeruginosa Exotoxin (EPA) and the Campylobacter protein AcrA to be suitable protein carriers, most preferably EPA. AcrA contains natural glycosylation sites whereas EPA needs to be modified to encode glycosylation sites. Preferably, EPA is modified to introduce two glycosylation sites directed to the Shigella OI antigen. More preferably, two consensus sequences are introduced as discussed in Example 10.

The amino acid sequence of EPA, as modified in an embodiment of this invention to contain two glycosylation sites, is provided as SEQ. ID NO.: 6 (with signal sequence) and SEQ. ID NO.: 7 (without signal sequence) in the Sequence Listing provided below. The glycosylation sites in each of SEQ. ID NO.: 6 and SEQ. ID NO.: 7 are denoted with an underline.

FIG. 1OB shows a schematic of a carrier protein, such as EPA onto which N-glycosylation sites can be designed as Step 3 in the production of a bacterial bioconjugate. N-glycosylation sites require introduction of the consensus sequences discussed previously, namely, insertion of D/E-X-N-Z-S/T sequons, wherein X and Z may be any natural amino acid except proline. We have found that such consensus sequences preferably are introduced through surface loops, by insertion rather than mutation and considering using flanking residues to optimize the operation of the N-glycosylation site.

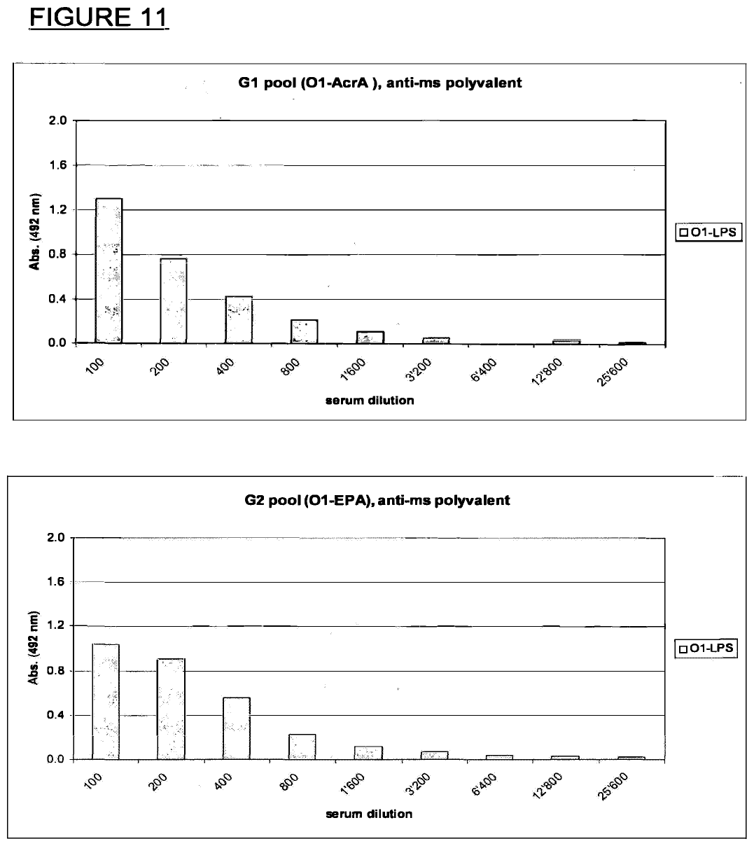

FIG. 11 shows bicoonjugates that elicit an immune response against Shigella OI polysaccharide in mice. OI-AcrA and OI-EPA was purified by affinity column and anionic exchange. The pure bioconjugate was injected into mice (n=10). Serum of mice that were immunized with OI-AcrA (top) or OI-EPA (bottom) three times (day 1, 21, 60) was pooled and analyzed by ELISA at day 70 for a sugar specific IgG response. The plates (Nunc, polysorb) were coated with LPS isolated from S. dysenteriae OI and incubated with the serum and anti mouse Polyvalent-HRP. Mice that received either conjugate developed an IgG response against the polysaccharide, confirming the presence of T-cell epitopes on the two protein carriers.

Consequently, the bacterial bioconjugates of the present invention show in vivo immunogenicity. In an embodiment, bacterial bioconjugates are capable of exhibiting: (1) a carbohydrate specific response; and (2) a carrier specific response or a similar response irrespective of the carrier protein. Moreover, an IgG specific response shows T-cell dependency of the immune response, such that memory of the response is expected.

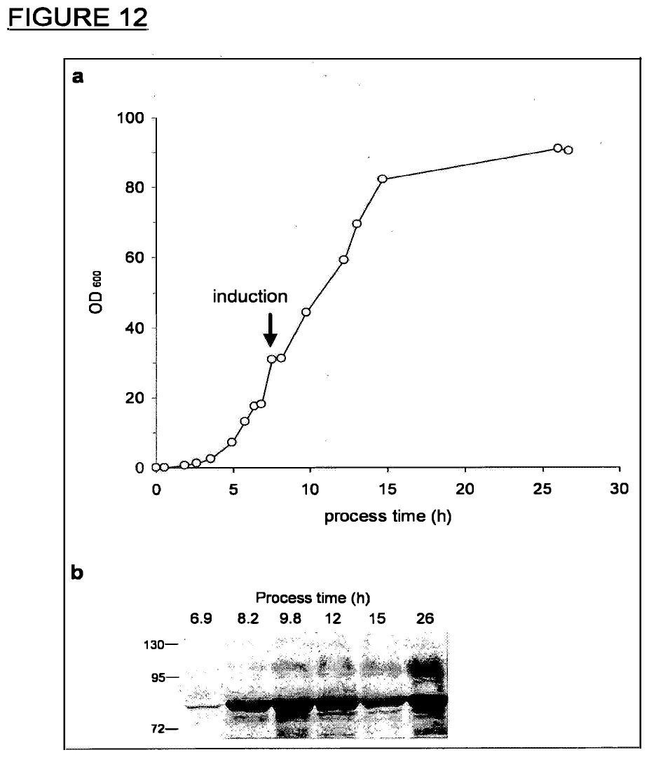

FIG. 12 reflects production of a Shigella OI bioconjugate, e.g., OI-EPA, in a bioreactor. E. coli cells expressing EPA, PglB and the OI-polysaccharide were grown in a bioreactor to OD₆₀₀=40 by two nutrient pulses. Expression of PglB and EPA was induced and the cells were grown overnight by linear feed of nutrients. The growth curve is depicted in the top panel. Whole cell extracts were separated by SDS-PAGE and expression and glycosylation of EPA was analyzed by immunodetection using a polyclonal antierserum that was raised against EPA (bottom). The cells efficiently glycosylate EPA at high cell density. The process is reproducible and leads to a total optical density (OD) of 90, which is a 45-fold increase compared to the shake flask culture. Consequently, scale-up is possible using the fed-batch process.

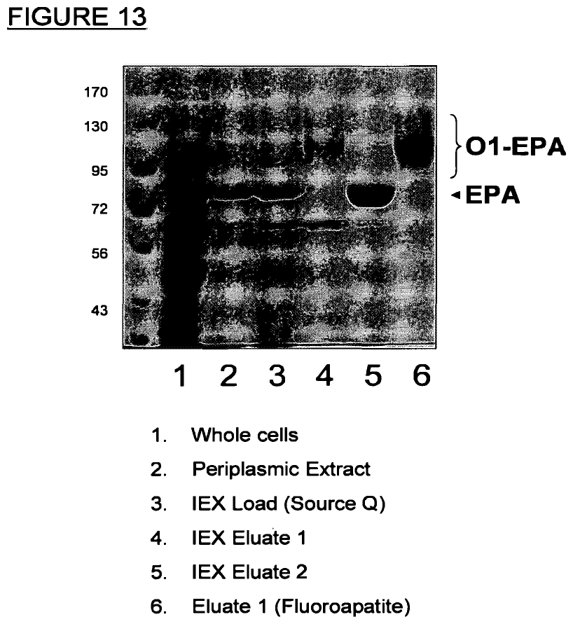

FIG. 13 shows an example of purification of OI-EPA. More specifically, FIG. 13 shows the fractionation and chromatographic purification of S. dysenteriae OI bioconjugate. E. coli cells expressing the OI-EPA were grown in the bioreactor to high cell density (See FIG. 12). The cells were pelleted by centrifugation and periplasmic proteins were extracted by osmotic shock. Periplasmic proteins were separated by anionic exchange (Source Q). Fractions enriched for OI-EPA were further purified by a second column (Fluoroapatite). The different fractions were separated by SDS-PAGE and the proteins were visualized by Coomassie Blue. Lane 1 shows whole cells extracts, lane 2 periplasmic proteins after osmotic shock, lane 3 periplasmic proteins loaded on anionic exchange, lane 4 and 5 eluates from anionic exchange and lane 6 OI-EPA eluate after the second purification column. This process allows the purification of OI-EPA at large scale. In this embodiment, the purification process is: (1) efficient (at >10 mg/L culture); (2) possible at large scale; and (3) compatible with Good Manufacturing Practices (GMP). Following such purification, the EPA-OI yield for glycerol-LB fed-batch was up to 200 mg/L, which is substantially higher than the yield for LB shake flask, which was 0.6 mg/L.

FIG. 14A shows a Western blot analysis of a series of specimens of AcrA-Shigella OI bioconjugates produced in an LB shake flask taken under various conditions, including pre-induction, 4 hours after induction, and 4 hours and 19 hours after induction under oxygen-limited circumstances. After extraction and purification, periplasmic proteins were separated by SDS-PAGE. AcrA and AcrA-Shigella OI bioconjugates were detected using anti-AcrA antibody and chemiluminescent detection via a secondary antibody. Loaded samples were normalized to culture OD₆₀₀ at time of sampling.

In summary, in one aspect, the technology of the present invention has been used to develop a vaccine against S. dysenteriae OI infection. For example, the polysaccharide of S. dysenteriae OI can be conjugated to EPA in E. coli. This is very beneficial since EPA previously has been successfully used in clinical trials with different conjugate vaccines. In the instant invention, the S″. dysenteriae OI bioconjugate was produced in a bioreactor at 31 scale. The cells were grown to high OD and the bioconjugate was extracted by osmotic shock. The bioconjugates were purified to 98% purity by anionic exchange and size exclusion chromatography. The bioconjugates were injected into different mice strains. After two as well as three injections, a sugar specific IgG response against the polysaccharide was detected using LPS from Shigella dysenteriae OI for analysis (FIG. 11). As expected, IgM specific response was elicited when the LPS was injected. The bioconjugates raised a specific IgG response against the polysaccharide isolated from S. dysenteriae. IgG response against the corresponding sugar antigen, which was chemically coupled to a carrier protein, has been shown to correlate with protection in humans.

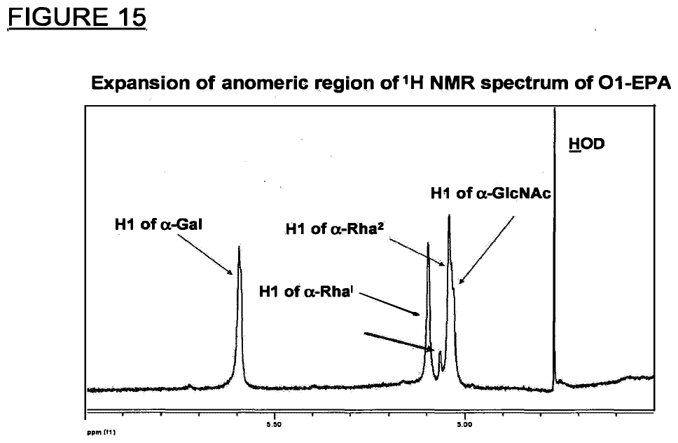

These results strongly suggest that our inventive E. coli strain is suitable for the potential production of an antigenic bacterial vaccine, such as an antigenic Shigella vaccine. In an embodiment, the EPA-Shigella bioconjugate was characterized intensively by different methods, like NMR, HPLC and MS. FIG. 15 shows the expansion of the anomeric region of IH NMR spectrum of an example of a & dysenteriae Serotype OI bioconjugate of the invention. The bioconjugate contains a sugar/protein ratio of 0.15, with 13.2 repeating units of the antigen being linked to the protein, and 1-2 sites being glycosylated. Two consensus sequences for glycosylation were introduced into EPA and about 20% of the protein is fully glycoslyated. The polysaccharide is linked via the reducing end to the protein carrier, therefore, the antigen epitopes of the polysaccharide are unmodified. In addition, the in vivo conjugation method attaches just the O-antigen repeating units to the protein, but no monosaccharides of the lipid A core are attached.

Using this technology, bacterial bioconjugates can be produced that are immunogenic. Genetic modifications can be made allowing in vivo conjugation of bacterial polysaccharides in desired proteins and at desired positions. For example, in an embodiment and as discussed above, the antigenic polysaccharide of S. dysenteriae OI can be expressed in E. coli and conjugated to two different protein carriers in vivo (i.e., EPA and AcrA). Both bioconjugates elicit a specific IgG response against the polysaccharide in mice. As another example, Table 1 below depicts different polysaccharide substrates for bacterial OSTs/OTases such as PglB that can be used in the in vivo method of the present invention for conjugating a protein carrier with the polysaccharide.

TABLE 1

| | |
|---|---|
| C. jejuni N-Glycan | 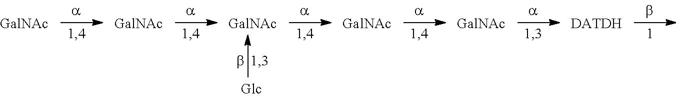 |
| Shigella dysenteriae O1 | 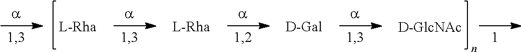 |
| Pseudomonas aeruginosa O11 | 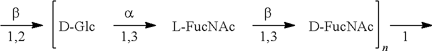 |
| E. coli O16 | 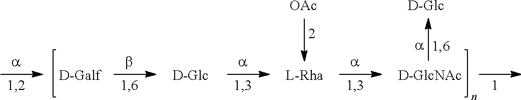 |

Table 2 below depicts yet additional different LPS polysaccharide substrates that could be utilized in the present invention with respect to various strains of Shigella and E. coli., as well as of Pseudomonas aeruginosa Oi I and Francisella tularensis.

TABLE 2

| | |
|---|---|
| Shigella dysenteriae O1 | 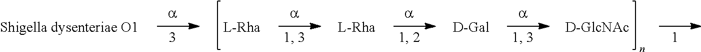 |
| S. flexneri 2a | 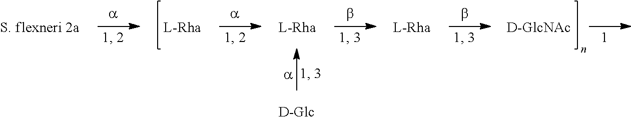 |
| S. flexneri 3a | 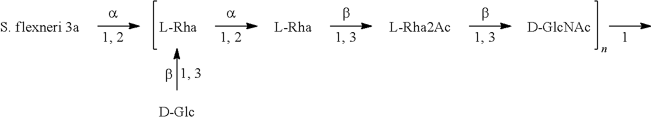 |
| S. flexneri 3b | 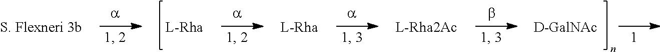 |
| S. flexneri 6 | 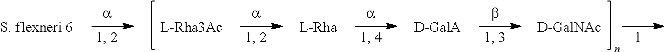 |
| S. sonnei | 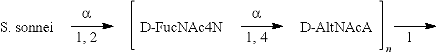 |
| E. coli O4:K52 (Ex PEC) | 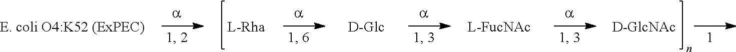 |
| E. coli O4:K6 (ExPEC) | 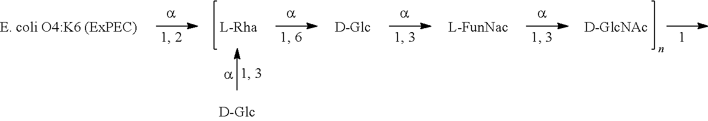 |
| E. coli O6:K2 (ExPEC) | 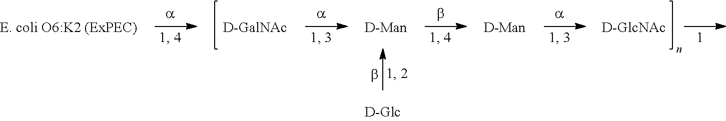 |
| E. coli O6:K54 (ExPEC) | 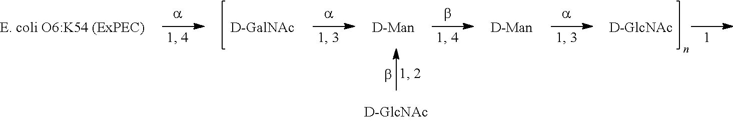 |
| E. coli O22 (Ex PEC) | 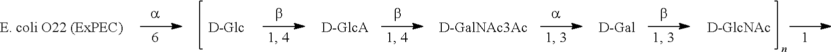 |
| E. coli O75 (ExPEC) | 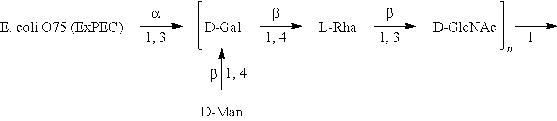 |
| E. coli O83 (ExPEC) | 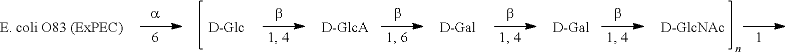 |
| E. coli O7 | 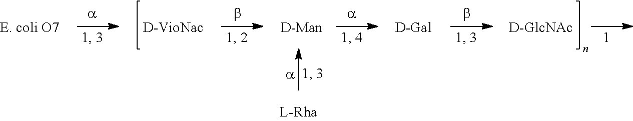 |
| E. coli O9 | 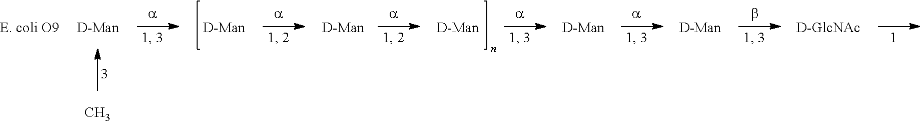 |
| E. coli O16 | 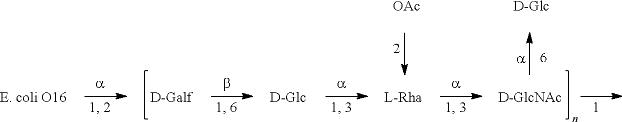 |
| E. coli O121 | 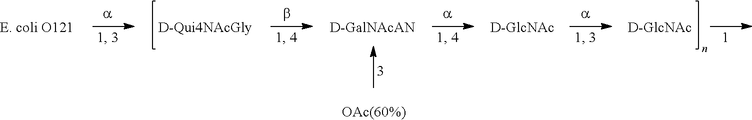 |
| E. coli O157 (EHEC) | 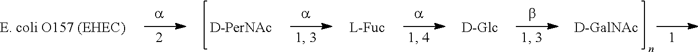 |
| Pseudomonas aeruginosa O11 | 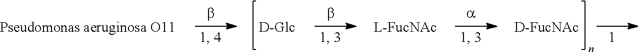 |
| Francisella tularensis | 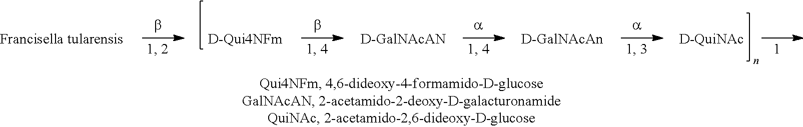 Qui4NFm, 4,6-dideoxy-4-formamido-D-glucose GaINAcAN, 2-acetamido-2-deoxy-D-galacturonamide QuiNAc, 2-acetamido-2,6-dideoxy-D-glucose |

For example, in a further embodiment of the invention, bioconjugate vaccines against E. coli can also be developed. E. coli is a well-known bacterial species. From a genetic and clinical perspective, E. coli strains of biological significance to humans can be broadly categorized as commensal strains, intestinal pathogenic strains and extraintestinal pathogenic E. coli (ExPEC). ExPEC strains can be part of the normal intestinal flora and are isolated in 1 1% of healthy individuals. They do not cause gastroenteritis in humans but their main feature is their capacity to colonize extraintestinal sites and to induce infections in diverse organs or anatomical sites. They are the main cause of urinary tract infections (UTI), are involved in septicemia, diverse abdominal infections and meningitis. Bacteremia can arise with a risk of severe sepsis. Severe sepsis due to ExPEC was associated with 41,000 estimated deaths in 2001. ExPEC strains have been susceptible to antibiotics; however more and more antibiotic resistant strains have evolved, both in hospital and in the community. This antimicrobial resistance is making the management of ExPEC infections more difficult; therefore, new vaccines would be an alternative strategy to prevent these infections.

In animal models, passive or active immunization against capsule, O-specific antigen and different outer membrane proteins have afforded protection against systemic infections and immunization with these different antigens are protective against urinary tract infections from ExPEC strains expressing these virulence factors. The serotypes 04, 06, 014, 022, 075 and 083 cause 75% of UTI. In one embodiment, the novel technology of the present invention can be used to develop a monovalent LPS bioconjugate including one antigen (e.g., serotype 06, one of the major serotypes) and even a multivalent LPS bioconjugate including these 6 antigens. For example, the gene cluster encoding for the enzymes that synthesize the O-antigen for ExPEC could be amplified and then expressed in the Shigella production strain.

The instant invention involves a highly efficient production process with high potential yields that can be used for industrial scale preparations in a cost-efficient process. This novel, cost efficient bioengineering approach to producing bioconjugate can be applied to other conjugates and for other applications. An additional feature of the invention involves a considerable simplification of the production of bacterial vaccines with high reproducibility and a potentially reduced risk of lot failures.

### Process for Manufacturing Conjugate Vaccine

It is now possible to engineer bacterial expression systems so that specific bioconjugates are produced that are biologically active. For example, the O-specific polysaccharide of S. dysenteriae has been conjugated to different protein carriers and the resulting bioconjugate has elicited a specific IgG response against the polysaccharide in mice. In an embodiment, the technology of the invention makes use of an oligosaccharyl transferase, for example, PglB of Campylobacter jejuni to couple bacterial polysaccharides (O antigens) in vivo to simultaneously express recombinant carrier proteins, yielding highly immunogenic bioconjugate vaccines.

A production process has been established that can be used on an industrial scale. This opens up the possibility that a multitude of various conjugate vaccines can be developed and manufactured using simple bacterial fermentation. The process has several advantages compared to the in vitro conjugation method depicted in the top panel of FIG. 6A. As it is a complete in vivo process, the cost and risk of failures are reduced significantly and the process is more reproducible. In addition, the consensus capture sequence allows the conjugation of polysaccharides to defined proteins at specific built-in sites, thereby facilitating regulatory acceptance and quality control. Finally, the development of conjugate vaccines is much faster since the process is simplified and requires only biotechnology tools. In addition, the in vivo conjugation process is suited for application where polysaccharide compositions prevent chemical cross-linking.

In an embodiment, the instant invention relates to the scaled-up production of recombinant glycosylated proteins in bacteria and factors determining glycosylation efficiency. For example, recombinant glycosylated proteins of the present invention can be made using the shakeflask process. Bioconjugates have previously been mainly produced in LB shake flask cultures. More preferably, in one aspect of the invention, a first fed-batch process can be used for the production of recombinant glycosylated proteins in bacteria. In a preferred manufacturing process, the aim is to achieve markedly increased final biomass concentrations while maintaining glycosylation efficiency and recombinant protein yield per cell and while maintaining simplicity and reproducibility in the process.

In one embodiment, bacterial bioconjugates of the present invention can be manufactured on a commercial scale by developing an optimized manufacturing method using typical E. coli production processes. First, one can use various types of feed strategies, such as batch, chemostat and fed-batch. Second, one can use a process that requires oxygen supply and one that does not require an oxygen supply. Third, one can vary the manner in which the induction occurs in the system to allow for maximum yield of product.

It has found been that, in contrast to expression of the carrier protein, the degree of N-linked glycosylation strongly reacts to changes in nutrient availability, type of carbon- and energy source, oxygen supply and time-point of induction. For example, in a fed-batch process, the addition of inducers to the batch and fed-batch cultures immediately leads to a 3-fold decrease in specific growth rate, indicating a high metabolic burden and/or stress due to synthesis of the carrier protein and membrane-bound oligosaccharyltransferase. Based on the inventors' finding of a recurring retardation of the appearance of glycosylated carrier protein compared to the non-glycosylated form after induction, it is concluded that glycosylation appears to be the rate-limiting step in bioconjugate biosynthesis.

Based on these results, in an example of an embodiment of the invention, the following process design for cultivation has been developed: fed-batch cultivation mode for achieving high cell densities; extended incubation after induction to facilitate maximal glycosylation; surplus nutrient supply (e.g., LB components yeast extract and tryptone) during biomass build-up until induction to provide a sufficient supply of building blocks for the production process; and glycerol as the main carbon and energy source to prevent catabolite repression and acetate formation. This bioprocess allows a 50-fold increase in yield compared to LB batch culture, paving the way towards a cost-effective production of conjugate vaccines in recombinant Escherichia coli. In this example, one can have oxic conditions throughout the production process, for example, achieved through oxygen-enriched aeration; however, low oxygen content is also feasible. Example 9 sets forth this example of a fed-batch process in greater detail. It should be recognized, however, that other processes may be used to produce the bacterial LPS bioconjugates of the present invention.

Consequently, in one embodiment of the invention, E. coli can be used for in vivo production of glycosylated proteins and is suitable for industrial production of glycosylated proteins.

The following examples serve to illustrate further the present invention and are not intended to limits its scope in any way.

### EXAMPLES

### Example 1: Selection of AcrA as Model Protein for Optimizing N-Glycosylation

To optimize the acceptor protein requirements for N-glycosylation detailed studies were performed on the C. jejuni glycoprotein AcrA (Cj0367c). AcrA is a periplasmic lipoprotein of 350 amino acid residues. It has been shown that secretion to the periplasm but not lipid-anchoring is a prerequisite for glycosylation (Nita-Lazar et al., 2005, supra). The signal for export can either be the native AcrA signal sequence or the heterologous PeIB signal when expressed in E. coli. Of the five potential λMinked glycosylation sequons (NI 17, N123, N147, N273, N274) the same two ones are used in C. jejuni and E. coli (N 123 and N273 (Nita-Lazar et al., 2005, supra)). AcrA was chosen as model because it is the only periplasmic N-glycoprotein of C. jejuni for which detailed structural information is available. Recently, the crystal structure of an AcrA homologue, the MexA protein from the Gram-negative bacterium P. aeruginosa, was published (Higgins et al., (2004). Structure of the periplasmic component of a bacterial drug efflux pump. Proc. Natl. Acad. Sci. USA 7Ofi 9994-9999). Both proteins are members of the so-called periplasmic efflux pump proteins (PEP, (Johnson, J. M. and Church, G. M. (1999). Alignment and structure prediction of divergent protein families: periplasmic and outer membrane proteins of bacterial efflux pumps. J. Mol. Biol. 287, 695-715)). The elongated molecule contains three linearly arranged subdomains: an α-helical, anti-parallel coiled-coil which is held together at the base by a lipoyl domain, which is followed by a six-stranded β-barrel domain. The 23-28 residues at the N-terminus and 95-101 residues in the C-terminus are unstructured in the crystals. MexA and AcrA protein sequences are 29.3% identical and 50% similar. Thus, the two proteins likely exhibit a similar overall fold.

### Example 2: Elucidation of the Primary Peptide Sequence that Triggers Glycosylation

It is known that lipoyl domains similar to MexA of P. aeruginosa and accordingly also in AcrA of C. jejuni form a compact protein that can be individually expressed in E. coli (reviewed by Berg, A., and de Kok, A. (1997). 2-Oxo acid dehydrogenase multienzyme complexes. The central role of the lipoyl domain. Biol. Chem. 378, 617-634). To check which acceptor peptide sequence was required for N-glycosylation by the pgl machinery in E. coli the lipoyl domain of AcrA was taken. It was used as a molecular scaffold to transport peptides of different lengths to the periplasm and present them to the pgl machinery in vivo.

Therefore, a plasmid coding for the lipoyl domain (Lip) was constructed and N-terminally fused to the signal sequence of OmpA (Choi, J. H., and Lee, S. Y. (2004). Secretory and extracellular production of recombinant proteins using Escherichia coli. Appl Microbiol Biotechnol 64, 625-635) and C-terminally to a hexa histag. Cloning was performed to place the gene expression under the control of the arabinose promoter. For the Lip domain borders amino acid positions were chosen that appeared at the same positions as the domain borders of the Lipoyl domain part in MexA. To test different peptides for their ability to accept an N-glycan stretches of the sequence were inserted between the two hammerhead-like parts of the Lip domain. The stretches consisted of sequences comprising the N-glycosylation site N 123 of C. jejuni AcrA. The resulting open reading frames consisted of the sequences coding for the OmpA signal sequence, the N-terminal hammerhead-like part of AcrA (D60-D95, the numbering of the amino acids refers to the mature AcrA polypeptide sequence numbering), the different stretches containing the native N 123 glycosylation site of AcrA (see below), the C-terminal hammerhead-like part of AcrA-Lip (L167-D210) and the C-terminal his-tag.

Construction of the plasmids was achieved by standard molecular biology techniques. Three stretches containing the native N 123 glycosylation site of AcrA of different lengths were inserted between the two halves of Lip resulting in three different ORFs:

Construct A contains AI 18-SI 30 resulting in a protein sequence of:

(SEQ. ID NO. 8)
MKKTAIAIAVALAGFATVAQADVIIKPOVSGVIVNKLFKAGDKVKKGQT
LFHEODOASKDFNRSKALFSQLDHTEIKAPFDGTIGDALVNIGDYVSAS
TTELVRVTNLNPIYADGSHHH HHH.

Construct B contains FI 22-EI 38 resulting in a protein sequence of:

(SEQ. ID NO. 9)
MKKTAIAIAVALAGFATVAOADVIIKPOVSGVIVNKLFKAGDKVKKGOT
LFIIEQDOFNRSKALFSQSAISQKELDHTEIKAPFDGTIGDALVNIGDY
VSASTTELVRVTNLNPIYADGS HHHHHH.

Construct C contains D 121-A 127 resulting in a protein sequence of:

(SEQ. ID. NO. 10)
MKKTAIAIAVALAGFATVAOADVIIKPQVSGVIVNKLFKAGDKVKKGQT
LFIIEQDQDFNRSKALDHTEIKAPFDGTIGDALVNIGDYVSASTTELVR
VTNLNPIYADGSHHHHHH.

The underlined stretches of sequence indicate the OmpA signal peptide, singly underlined residues were introduced for cloning reasons or to render the protein resistant to degradation. Bold: glycosylation site corresponding to N 123 of AcrA. Italics: hexa-histag. The corresponding genes were expressed under the control of the arabinose promoter in the backbone of the plasmid pEC415 (Schulz, H., Hennecke, H., and Thony-Meyer, L. (1998). Prototype of a heme chaperone essential for cytochrome c maturation. Science 281, 1197-1200).

To check which of the three stretches triggered glycosylation of the Lip proteins protein expression experiments were performed. E. coli Top 10 cells (Invitrogen, Carlsbad, CA, USA) carrying pACYCpgl or pACYC/?g/mut (Wacker et al., 2002, supra) and a plasmid coding constructs Ai B or C were grown in LB medium containing ampicillin and chloramphenicol up to an OD of 0.5 at 37° C. For induction 1/1000 volume 20% arabinose (w/v) solution was added and the cells were grown for another 2 hrs. The cells were then harvested by centrifugation and resuspended in 20 mM Tris/HCl, pH 8.5, 20% sucrose (w/v), 1 mM EDTA, 1 mM PMSF, and 1 g/l (w/v) lysozyme and incubated at 4° C. for 1 hr. Periplasmic extracts were obtained after pelleting of the spheroblasts and diluted with 1/9 volume (v/v) of 1O× buffer A (3 M NaCl, 0.5 M Tris/HCl, pH 8.0 and 0.1 M imidazole) and MgSO₄ added to 2.5 mM. Ni-affinity purification was performed on 1 ml Ni-Sepharose columns from Amersham Pharmacia Biotech (Uppsala, Sweden) in buffer A. Proteins were eluted in buffer A containing 0.25 M imidazole.

FIG. 1 shows Coomassie brilliant blue stained SDS-PAGE gel of the peak elution fractions from the Ni-purified periplasmic extracts. The expression analysis showed that construct B produced a prominent single protein species (FIG. 1, lane 1). Constructs A and C both lead, in addition to the prominent protein, to a second protein band with slower electrophoretic mobility (FIG. 1, lanes 2 and 3). That the heavier protein species was indeed glycosylated was proven by MALDI-TOF/TOF (not shown). The only amino acid missing in construct B but present in A and C was D121, the aspartate residue 2 positions N-terminally to the glycosylated N 123. This demonstrates that D 121 plays an important role for glycosylation by the OTase. To verify that D 121 is essential for glycosylation it was mutated to alanine in construct C. Expression analysis resulted in only one protein band (FIG. 1, lane 4), thus showing that DI 21 is important for glycosylation. Furthermore, the fact that an artificial peptide display protein can be glycosylated shows that a short peptide of the D/E-X-N-Y-S/T type contains all information for C. jejuni-borne N-glycosylation to occur.

### Example 3: Verification of Example 2: AcrA-D121A is not Glycosylated at N 123

To confirm the findings from the peptide display approach an aspartate to alanine mutation was inserted at position 121 (D121A, i.e. 2 residues before the glycosylated N 123) in the full length soluble version of the AcrA protein and it was tested whether the site N 123 could still be glycosylated in E. coli. In order to test this AcrA-D121A was expressed and its glycosylation status was analyzed. For the analysis an engineered AcrA was used. It differed from the original C. jejuni gene in that it contains the PeIB signal sequence (Choi and Lee, 2004, supra) for secretion into the periplasm and a C-terminal hexa histag for purification. It has been shown that this AcrA variant gets secreted, signal peptide-cleaved and glycosylated as the lipid anchored, native protein (Nita-Lazar et al., 2005, supra). The following is the amino acid sequence of the soluble AcrA protein:

(SEQ. ID NO. 11)
MKYLLPTAAAGLLLLAAOPAMAMHMSKEEAPKIOMPPQPVTTMSAKSED
LPLS/TYPAKLVSDYDVIIKPQVSGVIVNKLFKAGDKVKKGQTLFIIEQ
DKFKASVDSAYGQALMAKATFENASKDFNRSKALFSKSAISQKEYDSSL
ATFNNSKASLASARAQLANARIDLDHTEIKAPFDGTIGDALVNIGDYVS
ASTTELVRVTNLNPIYADFFISDTDKLNLVRNTQSGKWDLDSIHANLNL
NGETVQGKLYFIDSVIDANSGTVKAKAVFDNNNSTLLPGAFATITSEGF
IQKNGFKVPQIGVKQDQNDVYVLLVKNGKVEKSSVHISYQNNEYAIIDK
GLQNGDKIILDNF KKIQVGSEVKEIGAQLEHHHHHH

The underlined residues are the PeIB signal peptide, italics the hexa-histag, and bold the two natural glycosylation sites at N 123 and N273. A plasmid containing the ORF for the above protein in the pEC415 plasmid (Schulz et al., 1998) was constructed to produce pAcrAper.

The assay to test the glycosylation status of AcrA and mutants thereof (see below) was as follows: expression of AcrA was induced with 0.02% arabinose in exponentially growing E. coli CLM24 (Feldman et al., 2005, supra) cells containing the plasmid-bome/?g/ operon in its active or inactive form (pACYCpg/or pACYCpg/mut, see (Wacker et al., 2002, supra)) and a plasmid coding for AcrA (pAcrAper). After four hours of induction, periplasmic extracts were prepared as described above and analyzed by SDS-PAGE, electrotransfer and immunodetection with either anti-AcrA antiserum or R12 antiserum. The latter is specific for C. jejuni N-glycan containing proteins (Wacker et al., 2002, supra).

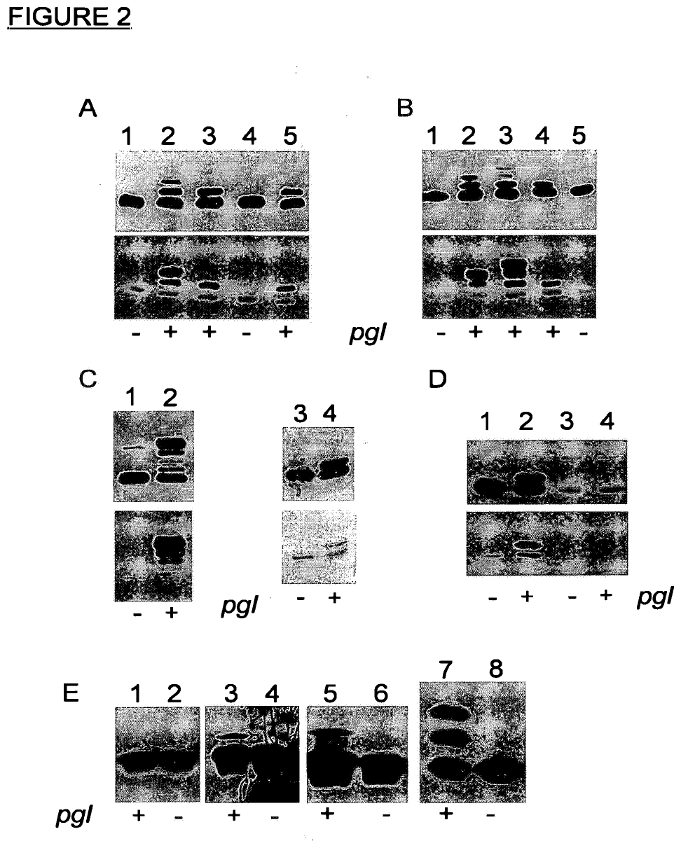

The first two lanes of FIG. 2A show AcrA in the absence and presence of a functional pgl operon. Only one band appears in the absence but three in the presence of the functional pgl operon (FIG. 2 A, top panel). These correspond to unglycosylated AcrA (lane 1) and un-, mono- and diglycosylated AcrA (lane 2). That the two heavier proteins in lane 2 were glycosylated was confirmed by the RI 2 western blot (lane 2, bottom panel). When the mutant AcrA-N273Q was expressed the same way, only the monoglycosylated AcrA was detected in presence of the functional glycosylation pgl operon (lane 3). Unglycosylated AcrA was detected in absence of the functional pgl locus (lane 4). Analysis of the mutant AcrA-D121A produced only two bands, one of them glycosylated (lane 5) as observed with AcrA-N273Q in lane 3. This means that D121 is essential for efficient glycosylation at position 123-125.

### Example 4: Introducing Artificial Glycosylation Sites into AcrA

To test if the introduction of an aspartate residue could generate a glycosylation site, AcrA mutants were generated in which the residue in the −2 position of the not used glycosylation sites in positions NI 17 and N 147 of soluble AcrA were exchanged for aspartate (FI 15D, T 145D). It was then tested whether the modified glycosylation sites could be glycosylated by the same assay as described in example 2. Both mutations were individually inserted either into the wildtype sequence of the soluble version of AcrA or in the double mutant in which both used glycosylation sites were deleted (N123Q and N273Q). Periplasms extracts of cultures induced for 4 hrs were prepared, separated by SDS page and analyzed by Western blotting (FIG. 2B). As controls the samples of wildtype glycosylated and non glycosylated AcrA were run on the same gel (lanes 1 and 2). The T145D mutation affected the −2 position of the natively not used glycosylation sequon NI 47-SI 49. Upon expression of AcrA-T145D Western blotting with anti AcrA antiserum resulted in four bands, the highest of them with slower electrophoretic mobility than the doubly glycosylated protein in lane 2 (lane 3 in FIG. 2B). The RI 2 blot confirmed that the fourth band was a triply glycosylated AcrA. Despite the low intensity towards anti AcrA the heaviest band gave the strongest signal with the glycosylation specific R12 antiserum. When the same mutant AcrA-T145D was expressed in the absence of the native N-glycosylation sequence (AcrA-N123Q-N273Q-T145D), only monoglycosylated AcrA was detected in the presence of a functional /?g/ operon (FIG. 2B, lane 4), that was missing in absence of a functional pgl operon (lane 5). This demonstrates that the heavier band in lane 4 was glycosylated. Hence, by simply introducing the T145D mutation an optimized glycosylation site was generated (DFNNS).

To further confirm that it is possible to introduce a glycosylation site by inserting an aspartate residue in the −2 position, the natively not used sites NI 17-SI 19 and N274-T276 were changed to optimize N-glycosylation. For this purpose further mutants were generated (FIG. 2C). Expression of AcrA-FI 15D-T145D in the above described system resulted in five protein species detected with the anti AcrA antiserum (lane 2). This is indicative for four glycosylates taking place on the same AcrA molecule. When the detection was performed with the C. jejuni N-glycan-specific R12 antiserum, a ladder of five bands was detected. The lowest faint band is unglycosylated AcrA because it is also present in the absence of glycosylation (lane 1), the highest results in a strong signal probably due to the five antigenic determinants in a fourfold glycosylated AcrA. Thus, the two introduced sites (at NI 17 and N 147) and the two natively used sites (N 123 and N273) are used and glycosylated by the pgl machinery. Expression of AcrA-N123Q-N273Q-N272D with and without the pgl operon demonstrated that a third artificially introduced glycosylation site, N274 (DNNST), was also recognized by the pgl operon (FIG. 2C, lanes 3 and 4).

The above experiments confirm the finding that the bacterial N-glycosylation site recognized by the OTase of C. jejuni consists partly of the same consensus as the eukaryotic one (N-X-S/T, with X #P) but, in addition, an aspartate in the −2 position is required for increasing efficiency. Furthermore, they demonstrate that it is possible to glycosylate a protein at a desired site by recombinantly introducing such an optimized consensus sequence.

### Example 5: Verification of Position −1 in the Optimized N-Glycosylation Sequence

A further experiment was performed to test whether the −1 position in the bacterial glycosylation site exhibits the same restrictions as the +1 position in eukaryotes (Imperiali, B., and Shannon, K. L. (1991). Differences between Asn-Xaa-Thr-containing peptides: a comparison of solution conformation and substrate behaviour with oligosaccharyl-transferase. Biochemistry 30, 4374-4380; Rudd, P. M., and Dwek, R. A. (1997). Glycosylation: heterogeneity and the 3D structure of proteins. Crit. Rev. Biochem. Mol. Biol. 32, 1-100). A proline residue at +1 is thought to restrict the peptide in such a way that glycosylation is inhibited. To test if a similar effect could also be observed in the −1 position a proline residue was introduced at that position of the first natively used site in a point mutant that had the second native site knocked out (AcrA-N273Q-F122P). The control expression of AcrA-N273Q showed a monoglycosylated protein in the presence of a functional /?g/ operon (FIG. 2D, lane 1 and 2). However, AcrA-N273Q-F122P was not glycosylated (FIG. 2D, lanes 3 and 4). This indicates that proline inhibited bacterial N-glycosylation when it constitutes the residue between the asparagine and the negatively charged residue of the −2 position.

Sequence alignments of all the sites known to be glycosylated by the C. jejuni pgl machinery indicate that they all comprise a D or E in the −2 position (Nita-Lazar et al., 2005, supra; Wacker et al., 2002, supra; Young et al., (2002). Structure of the N-linked glycan present on multiple glycoproteins in the Gram-negative bacterium, Campylobacter jejuni. J. Biol. Chem. 277, 42530-42539). Thus, it was established that the glycosylation consensus sequence for bacteria can be optimized by a negatively charged amino acid in the −2 position, resulting in D/E-X-N-Z-S/T, wherein X & Z; P.

### Example 6: N-Glycosylation of a Non-C. jejuni Protein

To demonstrate that the primary sequence requirement (optimized consensus sequence) is sufficient for N-glycosylation in bacteria, it was tested whether a non-C. jejuni protein could be glycosylated by applying the above strategy. Cholera toxin B subunit (CtxB) was employed as a glycosylation target. The corresponding gene was amplified from Vibrio cholerae in such a way that it contained the coding sequence of the OmpA signal sequence on the N-terminus and a hexahistag at the C-terminus, just the same as constructs A through C in example 1. The resulting DNA was cloned to replace construct A in the plasmids employed in example 1. A point mutation of W88 to D or a D insertion after W88 generated an optimized glycosylation site (DNNKT). The wildtype and W88D CtxB proteins containing the signal sequence and his-tag were expressed in E. coli Top 10 and other cell types in the presence and absence of the functional pgl locus from C. jejuni. When periplasmic extracts from Top 10 cells were analyzed by SDS-PAGE, electrotransfer and consecutive immunoblotting with a CtxB antiserum, only CtxB W88D produced a higher and thus glycosylated band in the pgl locus background (FIG. 2E, compare lanes 3 and 4). A consensus sequence (DSNIT) was also inserted by replacing G54 or Q56 of CtxB (the latter is denoted CtxB-Q56/DSNIT), i.e. in one of the loops that was reported to contribute to the ganglioside GM 1 binding activity of CtxB. Lanes 5 and 6 of FIG. 2E demonstrate that the engineered protein (exemplified by the construct which contains the peptide sequence DSNIT instead of Q56 expressed in Top 10 cells) produced a lower mobility and thus glycosylated band in glycosylation competent but not glycosylation-deficient cells when analyzed in the same way as described above. It was also demonstrated that a CtxB containing two manipulations, i.e. the insertion of D after W88 as well as DSNIT replacing Q56, was double-glycosylated in SCM7 cells (Alaimo et al., EMBO Journal 25: 967-976 (2006)) (panel E, lanes 7 and 8). The double-glycosylated protein CtxB shown in lane 7 was Ni²⁺ affinity-purified and analyzed by ESI− MS/MS after in-gel trypsinization according to standard protocols. The expected glycopeptides were detected confirming that bacterial N-glycosylation can also be directed to a non-C. jejuni protein by mutating or inserting the optimized consensus sequence according to the invention for bacterial N-glycosylation (not shown). Examples of other suitable exemplary E. coli strains for practicing the present invention are W3110, CLM24, BL21 (Stratagene, La Jolla, CA, USA), SCM6 and SCM7.

The amino acid sequence of the CtxB protein used here is indicated below (recombinant OmpA signal sequence underlined, hexa-histag italics, W88 bold):

(SEQ. ID NO. 12)
MKKTAIAIAVALAGFATVAOATPONITDLCAEYHNTOIHTLNDKIFSYT
ESLAGKREMAIITFKNGATFQVEVPGSQHIDSQKKAIERMKDTLRIAYL
TEAKVEKLCVWNNKTPHAIAAI SMANGSHHHHHH

### Example 7: Introduction of Artificial N-Glycosylation Sites into the C. jejuni Outer Membrane Protein. OmpHI

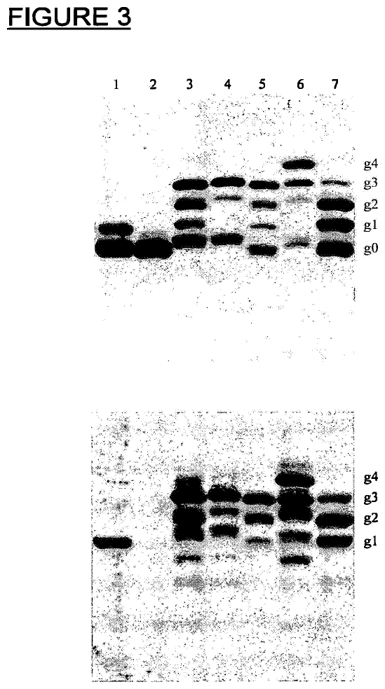

A potential application of the N-glycosylation in bacteria is the display of the glycan on the surface of a bacterial host cell in order to link the pheno- to the genotype and thereby select for specific genetic mutations. To demonstrate that N-glycans can be presented on outer membrane proteins the OmpHI protein was engineered in a way that it contained multiple optimized consensus sites according to the invention. The sites were engineered into loop regions of the protein as deduced from the known crystal structure (Muller, A., Thomas, G. H., Horler, R., Brannigan, J. A., Blagova, E., Levdikov, V. M., Fogg, M. J., Wilson, K. S., and Wilkinson, A J. 2005. An ATP-binding cassette-type cysteine transporter in Campylobacter jejuni inferred from the structure of an extracytoplasmic solute receptor protein. Mol. Microbiol. 57: 143-155). Previous experiments showed that the best glycosylation sequons were generated by the mutations V83T, K59N-G601-N61T, R190N-G191I-D192T and H263D-F264S-G265N-D266I-D267T. For surface display it was desired to evaluate different combinations of those introduced sites in order to establish the most N-glycan-specific sample. The combinations were generated in a wild type OmpHI encoding plasmid construct and tested in a similar manner as described for AcrA. FIG. 3 shows the analysis of various OmpHI variants harboring multiple glycosylation sequons in addition to the existing wild type sequon. OmpHI variants were generated with three (lane 3, 4, 5 and 7) and four glycosylation sequons (lane 6). A wild type OmpHI with only one glycosylation sequon and a mutant lacking the critical asparagine for glycosylation were also included in, the experiment. All variants tested here did not only demonstrate a high level of glycosylation efficiency but also that every glycosylation sequon was utilized.

The results were confirmed with Campylobacter N-glycan specific immuneserum (FIG. 3 lower panel).

The following is the amino acid sequence of the OmpHI protein of Campylobacter jejuni (strain 81-176) with attached myc tag in italics:

(SEQ. ID NO. 13)
MKKILLSVLTTFVAVVLAACGGNSDSKTLNSLDKIKONGWRIGVFGDKP
PFGYVDEKGNNQGYDIALAKRIAKELFGDENKVQFVLVEAANRVEFLKS
NKVDIILANFTQTPERAEQVDFCLPYMKVALGVAVPKDSNITSVEDLKD
KTLLLNKGTTADAYFTQDYPNIKTLKYDQNTETFAALMDKRGDALSHDN
TLLFAWVKDHPDFKMGIKELGNKDVIAPAVKKGDKELKEFIDNLIIKLG
QEQFFHKAYDETLKAHFGDDVKADDWIEGGKILEQKLZSEEDL

The native glycosylation site in the protein is bold, the signal sequence underlined.

### Example 8: Surface Display of N-Glycans from C. jejuni on OmpHI on the Outer Membrane of E. coli Cells

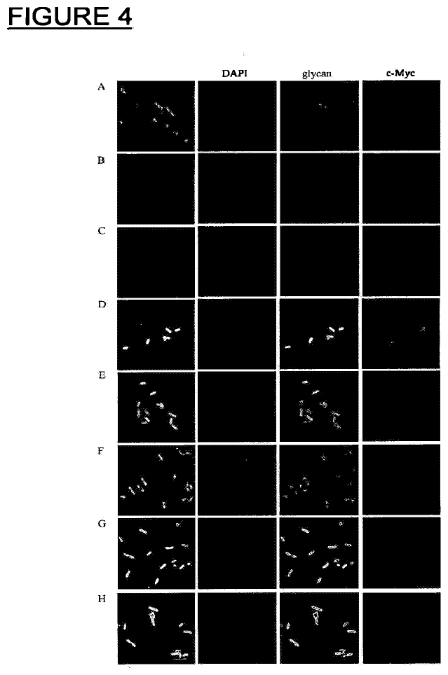

In order to answer the question whether multiple glycosylated OmpHI variants can be displayed on the surface of bacterial cells, immunofluorescence was performed on bacterial CLM24 or SCM6 (which is SCM7 ΔwaaL) cells expressing various OmpHI variants. A wild type OmpHI and a mutant lacking the critical asparagine for glycosylation were included in the experiment. In addition, a C20S mutant was constructed in order to retain the protein in the periplasm, thus serving as a control in the experiment. Immunostaining was carried out on the cells treated with paraformaldehyde. Paraformaldehyde fixes cells without destroying the cell structure or compartmentalization. The c-Myc- and N-glycan-specific immuneserum in combination with corresponding secondary antibodies conjugated to FITC and Cy3 were used to detect the protein (red fluorescence) and N-glycan (green) on the bacterial cell surface, respectively. Additionally, 4,6-diamino-2-phenylindole (DAPI, blue) was employed to stain for bacterial DNA to unambiguously differentiate between bacterial cells and cellular debris. When the cells expressing wild type OmpHI were stained, immunofluorescence specific to the protein as well as the N-glycan was detected (FIG. 4 A). When a mutant lacking the critical asparagine N139S was stained with both anti-Myc- and N-glycan-specific immuneserum only the protein but not glycan specific signals were obtained (panel 4 B) indicating specificity of the N-glycan-specific immune serum. When the protein was retained within the periplasm as in the C20S mutant, no protein specific, red immunofluorescence was detected indicating that the antibodies were unable to diffuse within the cell and were competent enough to detect any surface phenomenon (panel 4 C). Next, cells expressing multiple OmpHI variants different in glycosylate were stained: OmpHI^{KGN→NΠ}\, ^{HFGDD→DSNIT}^{_{\hat{ }\hat{ }}}_{4D) HFGDD→DSNIT AA} 4 D)

OmpHI^{RGD→NIT}, ^{HFGDD→DSNIT} (panel 4 E), ompHI^{KGN→NI}^{_{T′}}^{RGD→NIT} (panel 4 F), OmpHI^{V83T,KGN→NIT} (panel 4 G) and OmpHI^{ÂNIT RGD→NΓΓ HFGDD_→DSNIT}_{(pane, 4H) AU \hat{ }e}

OmpHI variants were double-stained indicating the presence of glycosylated protein on the bacterial surface. FIG. 4 is represented in grayscale, the first column is a merge picture of the other pictures of the same row.

FIG. 4 shows fluorescence microscopy of cells expressing various OmpHI variants. Cultures of E. coli strains CLM24 or SCM6 containing the expression plasmid for the wild type OmpHI and its variants were equalized to OD_{OOO} of 0.25/ml. Cells were washed two times with phosphate-buffered saline (PBS), pH 7.4 and 100 μl cell suspensions was dropped onto gelatinized glass slides and incubated at room temperature (RT) for 30 min inside a humidified chamber. All subsequent steps in the whole-cell immunofluorescence labeling were done at room temperature inside a humidified chamber. The unbound cells were removed and rest was fixed with 4% paraformaldehyde containing PBS for 30 min at RT. Importantly, paraformaldehyde is considered not to permeabilize cells but keeping the compartmentalization by membranes intact. Fixed cells were washed two times with PBS and resuspended blocking buffer containing 5% BSA in PBS. After blocking, the cells were incubated with anú-myc monoclonal mouse IgG (1:50, Calbiochem) and/or anti-glycan antiserum (1:4000) for 1 h in 100 μl of PBS containing 5% BSA. The cells were washed three times with 100 μl of PBS for 5 min each and incubated with secondary anti-rabbit antibody conjugated to FITC (1:250, Jackson Immunoresearch Laboratories) and/or anti-mouse antibody conjugated to Cy3 (1:250, Jackson Immunoresearch Laboratories) for 1 h in 100 μl of PBS containing 5% BSA. If required, 4, 6-diamino-2-phenylindole (DAPI) (Sigma) (0.5 μg/ml) was added at the time of secondary antibody incubation to stain for bacterial DNA. The secondary antibody was rinsed from the cells PBSi and coverslips were mounted on slides by using vectashield (Vector Laboratories) mounting medium and sealed with nail polish. Fluorescence microscopy was performed by the using an Axioplan2 microscope (Carl Zeiss). Images were combined by using Adobe Photoshop, version CS2. SCM6 cells expressing OmpHI (panel A), OmpHI^{N139S} (panel B), OmpHI^{C20S} (panel C), OmpHI^{KGN→NIT}, ^{HFGDD→DSNIT} (panel D), OmpHI^{RGD→NΓΓ·HFGDD-→DSNIT Â}, (panel E) OmpHI^{KGN→NIT RGD→NIT} (panel F), OmpHI^{V83T′KGN→N}^{_{ΓΓ}} (panel G), and OₘₚHi^{KGN_N}^{_{ΓΓ}}·^{RGD_N}^{_{ΓΓ′}}^{HFGDD→DSN}^{_{ΓΓ}} (panel H). The first column is a merge of the pictures in columns 2, 3, and 4 represented in greytones on black background. Column 2: blue fluorescence in greytones from DAPI stain, column 3: green fluorescence from glycan specific fluorescence, column 4: red fluorescence from anti-myc staining.

### Example 9: An Example of a Production Process for Shigella OI LPS Bioconjugate

This is an example of a production process; however, different conditions also lead to similar product formation.

### A. Production Process

E. coli strain W31 IOΔwaaL containing three plasmids expressing PglB, EPA and the enzymes for the biosynthesis of the Shigella OI polysaccharide was used for the production of the LPS bioconjugate. A single colony was inoculated in 50 ml LB medium and grown at 37° C. O/N. The culture was used to inoculate a 11 culture in a 21 bioreactor. The bioreactor was stirred with 500 rpm, pH was kept at 7.0 by auto-controlled addition of either 2 M KOH or 20% H₃PO₄ and the cultivation temperature was set at 37° C. The level of dissolved oxygen (pO2) was kept between 0 and 10% oxygen. The cells were grown in a semi defined glycerol medium containing Kanamycin to an OD600=15. The medium contained the following ingredients: 330 mM Glycerol, 10 g Yeast extract, 20 g Tryptone, 34 mM K₂HPO₄, 22 mM KH₂PO₄, 38 mM (NH₄)₂SO₄, 2 mM MgSO₄— 7 H₂O and 5 mM Citric acid. After an initial batch phase around 5 h, a first nutrient pulse was added to sustain fast biomass build-up (glycerol, tryptone and yeast extract). After an additional 1.5 h the culture reached an OD₆₀₀=30. At this timepoint a second nutrient pulse of glycerol and tryptone was added together with the required inducers 1% L-arabinose and 1 mM IPTG. In order to keep induction at maximum levels and supply further amino acids for recombinant protein synthesis, a linear nutrient/inducer feed (28.8 ml/h) was started with the addition this pulse. The feed was sustained until the end of the process. The bioreactor culture was harvested after a total of ˜24 h cultivation, when it should have reached an OD600 of ±80.

The production process was analyzed by Western blot as described previously (Wacker, M., et al., N-linked glycosylation in Campylobacter jejuni and its functional transfer into E. coli. Science, 2002. 298(5599): p. 1790-3.). After being blotted on nitrocellulose membrane, the sample was immunostained with the specific anti-EPA (Wacker, M., et al., N-linked glycosylation in Campylobacter jejuni and its functional transfer into E. coli. Science, 2002. 298(5599): p. 1790-3.). Anti-rabbit IgG-HRP (Biorad) was used as secondary antibody. Detection was carried out with ECL™ Western Blotting Detection Reagents (Amersham Biosciences, Little Chalfont Buchinghamshire).

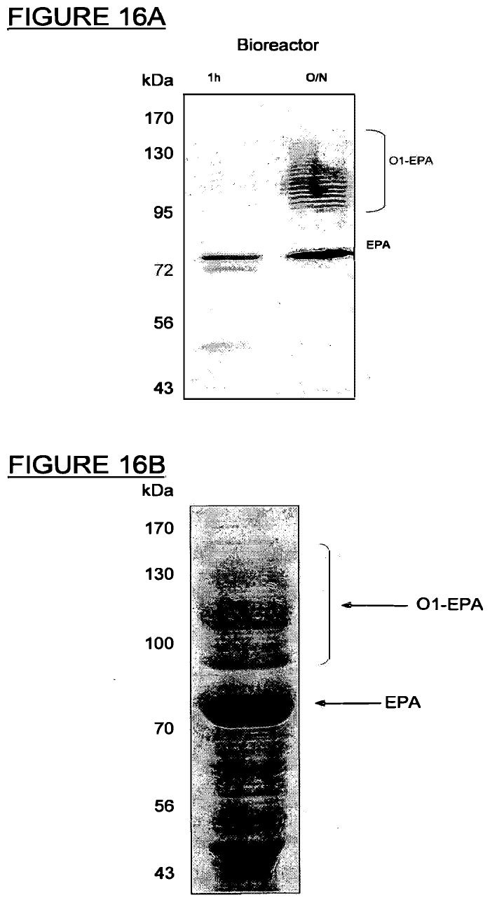

FIG. 16A shows proteins extracted of the Shigella OI LPS Bioconjugate (i.e., EPA-OI) from a fed-batch process that were normalized to biomass concentration (0.1 ODóoonm of cells/lane). The proteins were separated by SDS-PAGE transferred to Nitrocellulose membrane and visualized by rabbit anti EPA antibody. The induction time for PglB and EPA expression was 1 h and O/N.

### B. Periplasmic Protein Extraction

The cells were harvested by centrifugation for 20 min at 10,000 g and resuspended in 1 volume 0.9% NaCl. The cells were pelleted by centrifugation during 25-30 min at 7,000 g. The cells were resuspended in Suspension Buffer (25% Sucrose, 100 mM EDTA 200 mM Tris HCl pH 8.5, 250 OD/ml) and the suspension was incubated under stirring at 4-8° C. during 30 min. The suspension was centrifuged at 4-8° C. during 30 min at 7,000-10,000 g. The supernatant was discarded, the cells were resuspended in the same volume ice cold 20 mM Tris HCl pH 8.5 and incubated under stirring at 4-8° C. during 30 min. The spheroblasts were centrifuged at 4-8° C. during 25-30 min at 10,000 g, the supernatant was collected and passed through a 0.2μ membrane.

As shown in FIG. 16B, the periplasmic extract was loaded on a 7.5% SDS-PAGE, and stained with Coomasie to identify EPA and EPA-GI. EPA is a thick band that runs above the 70 kDa marker. OI-EPA (i.e., EPA-OI) runs as a leader between 100 and 170 kDa.

### C. Bioconjugate Purification

The supernatant containing periplasmic proteins obtained from 100,000 OD of cells was loaded on a Source Q anionic exchange column (XK 26/40˜180 ml bed material) equilibrated with buffer A (20 mM Tris HCl pH 8.0). After washing with 5 column volumes (CV) buffer A, the proteins were eluted with a linear gradient of 15 CV to 50% buffer B (20 mM Tris HCl+1 M NaCl pH 8.0) and then 2 CV to 100% buffer B. Protein were analyzed by SDS-PAGE and stained by Coomassie. Fractions containing OI-EPA were pooled. Normally the bioconjugate eluted at conductivity between 6-17 mS. The sample was concentrated 10 times and the buffer was exchanged to 20 mM Tris HCl pH 8.0.

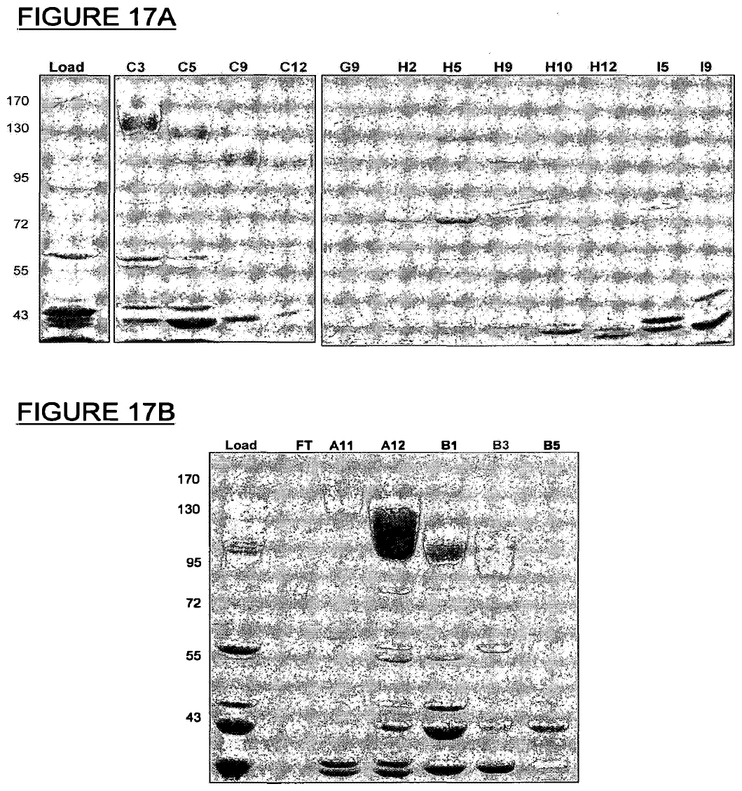

As shown in FIG. 17A, protein fractions from 1. Source Q were analyzed by SDS-PAGE and stained by Coomassie. Fractions CI to G9 contained OI bioconjugate and were pooled.

The OI-Bioconjugate was loaded a second time on a Source Q column (XK 16/20˜28 ml bed material) that has been equilibrated with buffer A: 20 mM Tris HCl pH 8.0. The identical gradient that was used above was used to elute the bioconjugate. Protein were analyzed by SDS-PAGE and stained by Coomassie. Fractions containing OI-EPA were pooled. Normally the bioconjugate eluted at conductivity between 6-17 mS. The sample was concentrated 10 times and the buffer was exchanged to 20 mM Tris HCl pH 8.0.

As shown in FIG. 17B, protein fractions from 2. Source Q column were analyzed on SDS-PAGE and stained by Coomassie. Fractions AI 1 to B3 containing OI bioconjugate were pooled.

The OI-Biconjugate was loaded on Superdex 200 (Hi Load 26/60, prep grade) that was equilibrated with 20 mM Tris HCl pH 8.0.

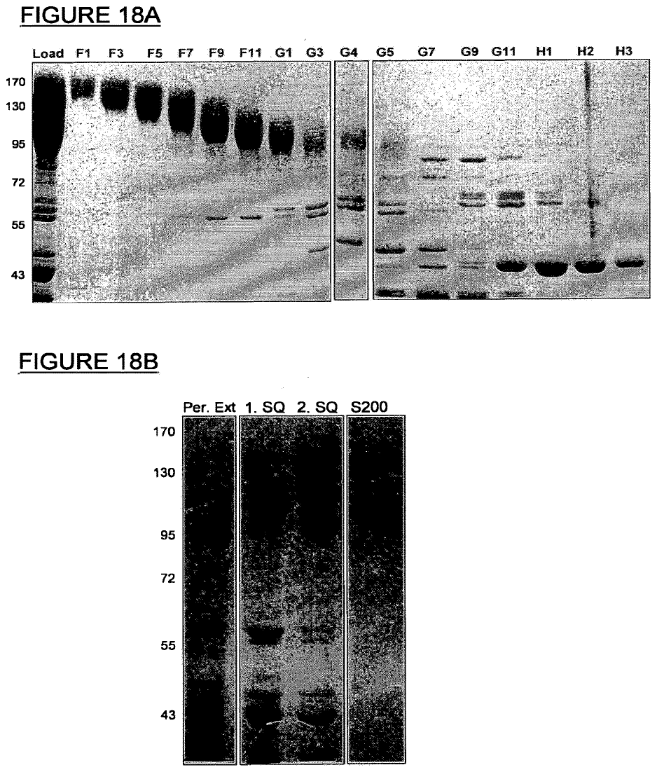

As shown in FIG. 18A, protein fractions from Superdex 200 column were analyzed by SDS-PAGE and stained by Coomassie stained. Fractions FI to FI 1 were pooled.

As shown in FIG. 18B, Shigella bioconjugate from different purification steps were analyzed by SDS-PAGE and stained by Coomassie. OI-EPA was purified to more than 98% purity using the process, showing that OI-EPA bioconjugate can be successfully produced using this technology.

### Example 10: Engineering of Exotoxin a of Pseudomonas aeruginosa for Glycosylation with Antigenic Carbohydrates

Exotoxin A of Pseudomonas aeruginosa (EPA) is a 67 kDa extracellulary secreted protein encoding mature 613 amino acids in its mature form and containing four disulfide bridges (CI 1-C15, C197-C214, C265-C287, C372-C379). To enable its glycosylation in E. coli, the protein must locate to the periplasmic space for glycosylation to occur. Therefore, a the signal peptide of the protein DsbA from E. coli was genetically fused to the N-terminus of the mature EPA sequence. A plasmid derived from pEC415 [Schulz, H., Hennecke, H., and Thony-Meyer, L., Prototype of a heme chaperone essential for cytochrome c maturation, Science, 281, 1197-1200, containing the DsbA signal peptide code followed by a RNase sequence was digested (NdeI to EcoRI) to keep the DsbA signal and remove the RNase insert. EPA was amplified using PCR (forward oligo was 5′-AAGCTAGCGCCGCCGAGGAAGCCTTCGACC (SEQ. ID NO. 14) and reverse oligo was 5′-AAGAATTCTCAGTGGTGGTGGTGGTGGTGCTTCAGGTCCTCGCGCGGCGG (SEQ. ID NO. 15)) and digested NheI/EcoRI and ligated to replace the RNase sequence removed previously. The resulting construct (pGVXN69) encoded a protein product with an DsbA signal peptide, the mature EPA sequence and a hexa-histag. Detoxification was achieved by mutating/deleting the catalytically essential residues L552VAE553 according to [Lukac, M., Pier, G. B., and Collier, R. J., Toxoid of Pseudomonas aeruginosa exotoxin A generated by deletion of an active-site residue, Infect Immun, 56, 3095-3098, 1988] and [Ho, M. M., et al., Preclinical laboratory evaluation of a bivalent Staphylococcus aureus saccharide-exotoxin A protein conjugate vaccine, Hum Vaccin, 2, 89-98, 2006] using quick change mutagenesis (Stratagene) and phosphorylated oligonucleotides 5′-GAAGGCGGGCGCGTGACCATTCTCGGC (SEQ. ID NO. 16) and 5′-GCCGAGAATGGTCACGCGCCCGCCTTC (SEQ. ID NO. 17) resulting in construct pGVXN70.

It is known that insertion of a pentapeptide sequence of the type D/E-Z-N-X-S/T into a suitable position results in glycosylation. To glycosylate EPA in E. coli cells, two different glycosylation sites were inserted into the previously described constructs according to the following description.

To insert a site at position 375, two steps were performed. First, quick change mutagenesis using oligos 5′-CCTGACCTGCCCCGGGGAATGCGCGG (SEQ. ID NO. 18) and 5′-CCGCGCATTCCCCGGGGCAGGTCAGG (SEQ. ID NO. 19) with pGVXN70 as a template resulted in a construct containing a single SmaI site at amino acid position 375 of EPA protein sequence by deleting three residues but otherwise keeping the starting protein sequence intact. In a second step, an insert composed of two complementary, phosphorylated oligonucleotides coding for (i) the previously deleted residues (when inserting the SmaI site), (ii) the pentapeptide glycosylation sequon and (iii) additional lysine residues flanking the consensus for optimization of glycosylation efficiency (as was found by further experiments) was ligated into this SmaI site (5′-GTCGCCAAAGATCAAAATAGAACTAAA (SEQ. ID NO. 20) and 5′-TTTAGTTCTATTTTGATCTTTGGCGAC (SEQ. ID NO. 21). The resulting construct was pGVXN137. To insert an additional glycosylation site in the construct at amino acid 240, a one step procedure using quick change mutagenesis with oligonucleotides 5′-CATGACCTGGACATCAAGGATAAT AATAATTCTACTCCCACGGTCATCAGTCATC (SEQ. ID NO. 22) and 5′-GATGACTGATGACCGTGGGAGTAGA ATTATTATTATCCTTGATGTCCAGGTCATG (SEQ. ID NO. 23) was applied on construct pGVXN137. The resulting construct thus contained various changes compared to the wild type EPA protein: two glycosylation sites, a DsbA signal peptide, detoxification mutation.

While this invention has been particularly shown and described with references to embodiments thereof, it will be understood by those skilled in the art that various changes in form and detail may be made therein without departing from the scope of the invention encompassed by the claims. Moreover, in instances in the specification where specific nucleotide or amino acid sequences are noted, it will be understood that the present invention encompasses homologous sequences that still embody the same functionality as the noted sequences. Preferably, such sequences are at least 85% homologous. More preferably, such sequences are at least 90% homologous. Most preferably, such sequences are at least 95% homologous.

1-29. (canceled)

30. An E. coli expression system for producing a bioconjugate vaccine against at least one bacterium comprising:
a nucleotide sequence encoding an oligosaccharyltrasferase (OST/OTase) which is PglB;
a nucleotide sequence encoding a protein carrier which is genetically detoxified Exotoxin of Pseudomonas aeruginosa (EPA) comprising at least two inserted consensus sequences, D/E-X-N-Z-S/T, wherein X and Z may be any natural amino acid except proline and a targeting polypeptide sequence; and
at least one antigenic polysaccharide synthesis gene cluster from at least one bacterium, wherein the antigenic polysaccharide synthesis gene cluster encodes a bacterial O-antigen from one or more strains of Shigella, Escherichia coli (E. coli), Pseudomonas aeruginosa and Francisella tularensis.

31. The expression system of claim 30, wherein the OST/OSTase has an amino acid sequence at least 90% identical to SEQ ID NO:2.

32. The expression system of claim 30, wherein the nucleotide sequence encoding the protein carrier encodes a protein carrier comprising a sequence at least 90% identical to SEQ ID NO:6.

33. The expression system of claim 30, wherein the at least one bacterial O-antigen is from extraintestinal pathogenic E. coli (ExPEC).

34. The expression system of claim 30, wherein the modified EPA has a sequence at least 90% identical to SEQ ID NO:7.

35. The expression system of claim 30, wherein the at least one bacterial O-antigen is from one or more of S. dysenteriae O1, S. flexneri 2a, S. flexneri 3a, S. flexneri 3b, S. flexneri 6 and S. sonnei.

36. The expression system of claim 30, wherein the bacterium a is pathogenic strain of a bacterium selected from the group consisting of Shigella, E. coli, Pseudomonas aeruginosa O11 and Francisella tularensis.

37. The expression system of claim 30, wherein the at least one bacterial O-antigen is from Pseudomonas aeruginosa O11.

38. An E. coli expression system for producing a Shigella bioconjugate vaccine comprising: a protein carrier comprising
a nucleotide sequence encoding a genetically detoxified Exotoxin of Pseudomonas aeruginosa (EPA) that has been modified to contain at least two consensus sequences D/E-X-N-Z-S/T, wherein X and Z is any natural amino acid except proline; and
a nucleotide sequence encoding at least one polysaccharide chain linked to an asparagine residue of the D/E-X-N-Z-S/T consensus sequence of the protein carrier and having the following structure:

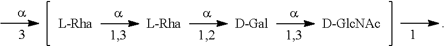

39. The expression system of claim 30, wherein the at least one bacterial O-antigen is from one or more of E. coli O4:K52 (ExPEC), E. coli O4:K6 (ExPEC), E. coli O₆:K2 (ExPEC); E. coli O6:K54 (ExPEC), E. coli O22 (ExPEC), E. coli O75 (ExPEC), E. coli O83 (ExPEC), E. coli O7, E. coli O9, E. coli O16, E. coli O121 and E. coli O157 (EHEC).
